(19) United States
(12) Patent Application Publication
(10) Pub. No.: US 2024/0331827 A1
(43) Pub. Date: Oct. 3, 2024

(54) Devices, Systems, and Methods for Affecting Adherence to Medication Protocols

(71) Applicants: THE REGENTS OF THE UNIVERSITY OF MICHIGAN, Ann Arbor, MI (US); SUBLIME, LLC, Okemos, MI (US)

(72) Inventors: Max Shtein, Ann Arbor, MI (US); Y. Eugene Shteyn, Cupertino, CA (US); Mohsen Moayer, New York, NY (US)

(21) Appl. No.: 18/034,546

(22) PCT Filed: Oct. 29, 2021
(86) PCT No.: PCT/US2021/057299

§ 371 Date: Apr. 28, 2023

Related U.S. Application Data
(63) Continuation-in-part of application No. 63/108,015, filed on Oct. 30, 2020.

(51) Int. Cl. G16H 20/10; G16H 40/67; G16H 80/00

(52) U.S. Cl. CPC G16H 20/10; G16H 40/67; G16H 80/00

(57) ABSTRACT

Disclosed herein are systems for tracking patient medication protocol adherence, the system comprising a computing device for tracking the amount of an active pharmaceutical ingredient (Al) to be administered to the subject using an augmented utensil comprising a layered Al delivery structure formed. Also disclosed are methods of customizing drug delivery, and augmented utensils and methods of making the same.

### BACKGROUND

### Technical Field

The present disclosure relates to methods and apparatuses for affecting adherence to medication protocols using medication delivery utensils and, more particularly, to augmenting utensils to include deposited films of organic compounds and film-coated substrates for affecting adherence.

### Description of Related Technology

Medications are typically prescribed to patients with specific adherence protocols detailing when and in what amounts to take a prescribed medication. Many of the patients are prescribed multiple medications, with complex adherence protocols, further detailing what order medications are to be taken.

A large proportion of the patients fail to follow the prescribed or recommended protocols for administering medication. In many patient populations, this problem is exacerbated due to intransigence or refusal (e.g., in case of patients suffering from dementia or other forms of mental impairment) and various conditions (e.g., dysphagia) reducing adherence to pill-centric drug regimens. Adding in that oftentimes multiple medications requiring multiple pills are prescribed, medication adherence is simply too cumbersome for too many.

The toll in human lives, the deleterious effect on quality of lives of patients and their loved ones, and the financial burden on individuals, families, and the healthcare system that are caused by poor adherence have far-reaching ramifications on society. Some analyses reveal that well over half of elderly patients do not adhere to their prescribed doses and/or schedules, with 200,000 elderly patients hospitalized annually due at least in part to non-adherence.

The problem of poor medication adherence will likely only worsen with the increasing push for tele-medicine and remote monitoring of patients. As monitoring technology advances, healthcare professionals are increasingly relying upon remote communications with patients to assess their wellbeing. However, there are no effective mechanisms for determining where a patient has adhered to their prescribed medication protocol. Most patients, the data shows, are unreliable in recounting their adherence. And for patients with dementia or other limiting conditions, confirmation is often not an option.

There is a need for effective mechanisms for monitoring of medication and/or supplement adherence protocols and for systems and methods to increase adherence to those protocols.

### SUMMARY

In response to these large and growing challenges of adherence, the present application provides utensils coated with medication and/or supplements, such that the active ingredient and/or supplement is ingested during a meal, which ameliorates the challenges presented by dementia, dysphagia, intransigence, refusal, and other impediments. Using one or more methods, including but not limited to solvent-free deposition [as shown by Shalev, Shtein et al., Nat Commun 8, 711 (2017), incorporated herein by reference], one or more coatings containing one or more active ingredients (Als) and/or inactive ingredients (excipients) are deposited onto utensils (including but not limited to forks, spoons, chopsticks, cups, sippy cups, lids of cups, lids of sippy cups, lids of reusable water bottles), optionally along with other information (e.g. patient name, dose, meal specification, color, design, etc.). The dose and release of the medication is tuned by adjusting the composition, thickness, and coated region of the layer(s). The invention also includes a system for specifying the type of delivery vehicle (such as type of utensil or container or cup) at the time of prescription or purchase request, any desired personalization, a subscription service for timely delivery of augmented utensils, and/or post-use assessment of medication quality, consumption patterns, with optional feedback on efficacy/adherence. Example benefits of this approach include increased adherence and substantially improved sanitation, as in the case of aseptic coating and packaging of the coated utensil. The latter is particularly important in light of the current COVID-19 pandemic, impacting long-term care facilities heavily; this invention provides for an easy means of offering individually packaged utensils, selectively coated with medicine, reducing instances of physical contact between potentially infected caregivers and the medicines, food, and drink ingested by patients.

In one aspect, described herein is a system for tracking patient medication protocol adherence, a computing device having one or more processors and one or more memories, the computing device communicatively coupled to a communication network, the computing device being configured to allow one or more remote user devices to access the computing device via the communication network; the computing device being further configured to, provide an interface of an application to the one or more remote user devices, receive, via the interface, identification data on a subject, the identification data including a pathology data for the subject, determine from the identification data a desired medication protocol for the subject, and convert the desired medication protocol into augmented utensil configuration data for the subject, the augmented utensil configuration data comprising (i) a desired amount of an active pharmaceutical ingredient (Al) to be administered to the subject using an augmented utensil comprising a working end having layered Al delivery structure formed thereon and (ii) augmented utensil data and/or usage data and/or patient data containing at least one parameter of the augmented utensil; the computing device being communicatively coupled, through the communication network, to a detection stage configured to analyze the augmented utensil and detect an amount of used Al, the computing device being further configured to, receive from the detection stage the amount of used Al and compare the amount of used Al to the desired amount of the Al stored in the augmented utensil configuration data, and from the comparison of the amount of used Al to the desired amount of the Al, determine an adherence score indicating a predicted adherence of the subject to the medication protocol. In some examples, the identification data comprises usage pattern, time of day, location, one or more Als to be taken, a medication delivery time frame for each of the one or more Als, preferred taste profile(s), counterindications, co-prescriptions, dosage, expiration dating information, storage instructions, safety instructions, prescribed regimen, regimen starting time and date, regimen ending time and date, patient gender, body weight, height, body mass index, place of administration, manner of administration, administration restrictions, temperature sensitive APIs restricted from certain utensils, preferred foods, counter-indicated foods, person responsible for administration, patient address, service subscription information, and/or service renewal information. In some examples, the augmented utensil data identifies a number of layers of an Al layered structure data of the utensil, identifies one or more excipient layers of the utensil, identifies a flavor masking layer of the utensil, identifies a delivery delay layer of the utensil, identifies a tracking ingredient of the utensil, identifies a non-API material of utensil, and identifies a pattern of locations of the Al on the utensil. In some examples, the augmented utensil data identifies a shape of a working end of the utensil. In some examples, the augmented utensil data identifies a location of the API on a working end of the utensil. In some examples, the computing device is further configured to compare the amount of used Al to the desired amount of the Al stored using a medication protocol model including one or more data elements of the augmented utensil configuration data. In some examples, the one or more remote user devices comprises a patient user device, and the computing device is configured to authenticate the patient user device. In some examples, the one or more remote user devices comprises a prescriber user device, and the computing device is configured to authenticate the prescriber user device. In an example, the computing device is configured to convert the desired medication protocol into augmented utensil configuration data for the subject: by accessing a listing of available augmented utensil models each having a different augmented utensil configuration data; ranking the available augmented utensil models according to a list of patient preferences, caretaker preferences, patient history, popularity across relevant demographic groups, preferred foods, preferred drinks, preferred administration methods, and dosage; and identifying a highest ranked available augmented utensil model. In an example, the computing device is configured to perform a validation on the detection stage to confirm the augmented utensil analyzed by the detection stage corresponds to the augmented utensil data. In an example, the computing device is configured to send a report of the adherence score to an external computing device and/or store the adherence score as patient data, facility data, and/or caretaker data.

In another aspect, described herein is a method of customizing drug delivery, the method comprising: receiving, at one or more processors, a drug delivery regimen profile for a subject, wherein the drug delivery regimen profile comprises at least one of one or more instances of usage pattern, time of day, location, one or more Is to be taken, a medication delivery time frame for each of the one or more Als, preferred taste profile(s), counterindications, co-prescriptions, dosage, expiration dating information, storage instructions, safety instructions, prescribed regimen, regimen starting time and date, regimen ending time and date, patient gender, body weight, height, body mass index, place of administration, manner of administration, administration restrictions, temperature sensitive Als restricted from certain utensils, preferred foods, counter-indicated foods, person responsible for administration, patient address, service subscription information, and/or service renewal information; accessing a database identifying a plurality of available active pharmaceutical ingredients (Als) and excipients, with corresponding information regarding the molecular weight of the Al, chemical structure, chemical database reference number(s), solubility, enthalpy of vaporization, vapor pressure, melting point, thermal decomposition point, viscosity, hardness, taste profile, counterindications, co-prescriptions, dosage, expiration dating information, storage instructions, safety instructions, associated billing codes, prescribed regimen for a given patient, regimen starting time and date, regimen ending time and date, patient gender, body weight, height, body mass index, genetic information, place of administration, manner of administration, administration restrictions, temperature sensitive APIs restricted from certain utensils, preferred foods, counter-indicated foods, person responsible for administration, patient address, service subscription information, and/or service renewal information; determining from the drug delivery regimen profile and the database an Al delivery structure comprising one or more of the available active Als and/or excipients; determining a configuration and/or Al pattern based on the drug delivery regimen; and depositing the Al delivery structure to a working end of a utensil having an elongated handle, the Al delivery structure being deposited according to the configuration and/or Al pattern.

In another aspect, described herein is a system for tracking patient medication protocol adherence, the system comprising: a utensil to which the active ingredient (Al), optionally combined with excipient(s), is directly or indirectly affixed or coated or layered onto, and subsequently removable from, enabling direct oral delivery of Al to the subject, as the subject may eat or drink using said utensil; a detection stage configured to receive the utensil and detect a usage measurement of and/or from the utensil; one or more processors; and one or more memories storing instructions that, when executed, cause the one or processors to: determine a desired amount of the Al to be provided to the subject orally based on subject profile data; determine an initial amount of the Al on the utensil; determine, from the usage measurement, an actual amount of the Al remaining on the utensil or an actual amount of the Al removed from the utensil; and determine a projected patient regimen adherence from a comparison of the actual amount of the Al remaining on the utensil or an actual amount of the Al removed from the utensil and the desired amount of the Al to be provided to the subject. In an example, the utensil comprises: an elongated handle end to be held in place during provision of the API to the subject; and a working end coupled to and extending from the handle end, the working end shaped to deliver the Al to the subject orally, the working end having surface onto which, directly or indirectly, the Al, and optionally the excipient(s), is or coated or layered. In an example, the utensil is a container. In an example, the utensil is a fork, spoon, blade chopstick, cup, sippy cup, lid of cup, lid of sippy cup, or lid of a reusable water bottle.

In another aspect, described herein is a system for tracking patient medication protocol adherence, the system comprising: a utensil to which the active ingredient (Al), optionally combined with excipient(s), is directly or indirectly affixed or coated or layered onto, and subsequently removable from, enabling direct oral delivery of Al to the subject, as the subject may eat or drink using said utensil; a detection stage configured to receive the utensil and detect a usage measurement of and/or from the utensil; one or more processors; and one or more memories storing instructions that, when executed, cause the one or processors to: determine a desired amount of the Al to be provided to the subject orally based on subject profile data; determine an initial amount of the Al on the utensil; determine, from the usage measurement, an actual amount of the Al remaining on the utensil or an actual amount of the Al removed from the utensil; and determine a projected patient regimen adherence from a comparison of the actual amount of the Al remaining on the utensil or an actual amount of the Al removed from the utensil and the desired amount of the Al to be provided to the subject.

In another example, described herein is a utensil comprising: an active ingredient (Al), optionally combined with excipient(s), is directly or indirectly affixed or coated or layered onto, and subsequently removable from, a body of the utensil enabling direct oral delivery of Al to subject, as the subject may eat or drink using said utensil.

In another aspect, described herein is a utensil (which may also be a container, by way of non-limiting example, a cup) comprising: an active ingredient (Al), optionally combined with excipient(s), is directly or indirectly affixed or coated or layered onto, and subsequently removable from, enabling direct oral delivery of Al to the subject, as the subject may eat or drink using said utensil. The utensil may optionally include a layered Al delivery structure with an indicator layer comprising a color indicator or symbology indicator, the indicator layer being at least partially covered by the Al film layer such that upon provision of the Al to the subject orally, the indicator layer becomes exposed or further exposed. Examples of the symbology indicator include but are not limited to a bar code, QR code, speckle pattern, grid, and others. The utensil also may optionally include a layered Al delivery structure further comprising a tracking layer that produces a signal when removed from the utensil or ingested by the subject. The utensil further may optionally include a layered Al comprising a delivery structure with a tracking layer that in a first state of the utensil is prevented from detection by the Al delivery structure and that in a second state of the utensil corresponding to removal of at least a portion of the Al is detectable. The layered Al delivery structure on the utensil may be layered or deposited on the lid of a container, such as a cup or sippy cup lid, or the cup itself, or the leading end of the container or utensil.

In another aspect, described herein is a utensil usage stage comprising: a housing; a sample area for positioning at least the working end of the utensil of claim 1 for inspection; a detection stage positioned to detect a usage indication signal from the utensil; and a processing stage comprising at least one processor and at least one memory, the processing stage being configured to determine from the usage indication signal detected by the detection stage at least one of absence of the Al on the utensil, presence of the Al on the utensil, an amount of Al absent from the utensil, an amount of Al present on the utensil.

Further aspects and advantages will be apparent to those of ordinary skill in the art from a review of the following detailed description, taken in conjunction with the drawings. The description hereafter includes specific embodiments with the understanding that the disclosure is illustrative, and is not intended to be limited to the specific embodiments described herein.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 shows medication protocol adherence system 100, in an example.

FIG. 2 shows an implementation of the medication protocol adherence system 100 with a medication adherence cloud platform 200 and example user station 202, in this example a patient station, in a first cloud-based configuration 201.

FIG. 3 shows an example process 300 for predicting medication protocol adherence as may be performed by the medication protocol adherence system 100 and/or the medication adherence cloud platform 200.

FIG. 4 shows the medication adherence cloud platform 200 communicatively coupled to augmented utensil fulfillment station 500 through the network 104 in a second cloud based configuration 401.

FIG. 5 shows the medication adherence cloud platform 200 communicatively coupled to an augmented utensil fulfillment station 500 for augmented utensils and/or packaging augmented utensils during manufacturing, including, in some examples through fully or partially automated ways under control of the cloud platform 200.

FIG. 6 shows a process 600 for generating an augmented utensil using the cloud-based configuration 501, in an example.

FIG. 7 shows an example detailed process 700 for generating an augmented utensil.

FIG. 8 shows a utensil 800 includes an elongated handle end 802 and a working end 804.

FIGS. 9A-C show a utensil where the working end includes an inner surface 806 (also termed an upper surface) and an outer surface 808 (also termed a lower surface) FIG. 9A shows an Al layered structure 810 is formed on the outer surface 808 and contains one or more Als corresponding to a medication protocol and as determined by a medication adherence platform system. FIG. 9B shows another configuration, with an Al layered structure 812 formed on the inner surface 806. FIG. 9C shows yet another pattern with an Al layered structure 814 that as Al layers formed on both the outer surface 808 and the inner surface 806.

FIGS. 10A and 10B show a cross section of a portion of the working end 804 with an Al layered structure formed thereon. FIG. 10A shows an Al layered structure 816 is formed of only one or more Als. FIG. 10B shows an Al layered structure 818 contains one or more Als and a non-Al in the form of an excipient agent within the structure.

FIG. 11 shows a multi-layered Al delivery structure 820 having an indicator layer 822, an adhesion control layer 824, an Al layer 826, a protective layer 828, and a taste modification layer 830, in an example.

FIG. 12 shows an Al layered delivery structure 850 may have an Al layer that contains an Al at patterned locations 852 and where a non-Al material 854 extends between the patterned locations 852.

FIG. 13 shows three different augmented utensil configuration patterns, a first where the Al delivery structure covers the entire working end, a second where the Al delivery structure coves a leading portion of the working end, and a third where different layers forming the Al delivery structure are formed at different locations of the working end.

FIG. 14 shows an augmented utensil showing indicator layers with a text descriptor of the Al.

FIG. 15 shows an example detection station 900 for detecting an amount of Al consumed on the utensil 800. The station 900 includes a sample area 902 configured to hold the utensil 800, for example, using a mount in the sample area configured to hold the elongated handled end and/or the working end. An activation stage 904 is positioned above the sample area 902 to examine the utensil and detect an amount of Al removed from and/or remaining on the utensil 800.

FIG. 16 shows an example where the activation stage comprises two parts an activator 914 and a detector 916, in accordance with an example.

FIG. 17 shows an example process in accordance with techniques herein, in particular an example adherence monitoring and improvement process, as may be performed by a data capture engine implemented by an example system herein, such as the computing device 102 or the medication adherence cloud platform 200.

FIG. 18 shows an example subscription-based access method, in accordance with an example.

FIG. 19 shows an example implementation of a system 1900 implementing techniques herein, according to an example.

FIG. 20 is a schematic of an example vapor deposition device, in accordance with an example. A drug vapor (API vapor) is jetted into a reservoir (left) with drugs and/or with solvents, wherein the compound vapor dissolves (right). A valve allows controlled introduction of the dissolved compound into a liquid handler, which can allow the compound to be introduced onto a utensil.

FIG. 21 is a schematic of the use of the vapor deposition device of FIG. 20 to produce stocks of compounds in solution at a controlled concentration. Drug vapor (API vapor) is jetted into a reservoir (left) with drugs and/or with solvents, wherein the drug vapor dissolves (right). This produces a high concentration “local stock solution” which can be redistributed by a liquid handler into well plates and further diluted to a desired concentration.

FIG. 22 is a schematic of the use of the vapor deposition device of FIG. 20 to produce deposit films of compound on a utensil: (A) drug vapor (e.g., an API in vapor form) is jetted into a compartment to produce a solid film. (B) the drug vapor can also be jetted directly onto the surface of the utensil to form a film. (C) drug vapor can also be deposited on a post to produce a solid film on the post. (D) drug vapor can also be deposited on crystals or as part of a crystal growing process.

FIG. 23 is a schematic of the use of the vapor deposition device of FIG. 20 to produce (A) discrete patterns of a drug (e.g., an API) on a utensil or (B) a continuous gradient of drug(s) across the utensil.

### DETAILED DESCRIPTION

In certain aspects, described herein are systems and methods of increasing a subject's adherence to medication protocols using a utensil having a substrate affected with one or more active ingredients, such as active ingredients (Als) or supplements for provision to the subject, where the utensil includes a tracking agent that allows a subsequent detection stage to determine an amount of Al delivered to the subject, from which adherence to the medication protocol is determined. Various examples herein discuss use of an Al and/or supplement, both of which may be collectively considered active ingredients in the present disclosure. Furthermore, references herein to an Al or a class of Als or to a supplement or a class of supplements are intended to include references to any other type of active ingredient contemplated herein. Furthermore, references herein to active ingredients are intended to contemplate examples that use inactive ingredients instead.

In an aspect, a system is provided for tracking patient medication protocol adherence. The system may include a computing device having one or more processors and one or more memories. The computing device may be communicatively coupled to a communication network, and configured to allow one or more remote user devices to access the computing device via the communication network. The computing device may be further configured to, provide an interface of an application to the one or more remote user devices, receive, via the interface, identification data on a subject, the identification data including a pathology data for the subject. The computing device may determine from the identification data a desired medication protocol for the subject and convert the desired medication protocol into augmented utensil configuration data for the subject. The augmented utensil configuration data may include (i) a desired amount of an active ingredient (Al) to be administered to the subject for treating the pathology using an augmented utensil comprising a working end having layered active pharmaceutical ingredient (Al) delivery structure formed thereon and (ii) augmented utensil data containing at least one parameter of the augmented utensil. Further, the computing device may be communicatively coupled, through the communication network, to a detection stage configured to analyze the augmented utensil and detect an amount of used Al. The computing device may be further configured to, receive from the detection stage the amount of used Al and compare the amount of used Al to the desired amount of the Al stored in the augmented utensil configuration data, and from the comparison of the amount of used Al to the desired amount of the Al, determine an adherence score indicating a predicted adherence of the subject to the medication protocol.

In another aspect, described herein is a method of customizing drug delivery. The includes: receiving, at one or more processors, a drug delivery regimen profile for a subject, which may include one or more instances of usage pattern, time of day, location, and/or other parameters of administration, such as amount of one or more Als to be taken, a medication delivery time frame for each of the one or more Als, preferred taste profile(s), counterindications, co-prescriptions, dosage, expiration dating information, storage instructions, safety instructions, prescribed regimen, regimen starting time and date, regimen ending time and date, patient gender, body weight, height, body mass index, place of administration, manner of administration, administration restrictions (e.g. temperature sensitive Als restricted from certain utensils), preferred foods, counter-indicated foods, person responsible for administration, patient address, service subscription information, and/or service renewal information. Further the method includes accessing a database identifying a plurality of available active pharmaceutical agents (Als) and excipients, with corresponding information regarding the molecular weight of the Al, chemical structure, chemical database reference number(s), solubility, enthalpy of vaporization, vapor pressure, melting point, thermal decomposition point, viscosity, hardness, taste profile, counterindications, co-prescriptions, dosage, expiration dating information, storage instructions, safety instructions, associated billing codes, prescribed regimen for a given patient, regimen starting time and date, regimen ending time and date, patient gender, body weight, height, body mass index, genetic information, place of administration, manner of administration, administration restrictions (e.g. temperature sensitive Als restricted from certain utensils), preferred foods, counter-indicated foods, person responsible for administration, patient address, service subscription information, and/or service renewal information. The method further includes determining from the drug delivery regimen profile and the film layer database a layered Al delivery structure comprising one or more of the available active Als and excipient layers. The method further includes determining a layering configuration and/or Al pattern based on the drug delivery regimen. Further the method includes depositing the Al delivery structure to a working end of a utensil having an elongated handle, the Al delivery structure being deposited according to the layering configuration and/or Al pattern.

In another aspect, described herein is a system for tracking patient medication protocol adherence. The system includes a utensil having an elongated handle end to be held in place during provision of an active pharmaceutical ingredient (Al) to a subject; and a working end coupled to and extending from the handle end, the working end shaped to deliver the Al to the subject orally, the working end having an upper surface and an opposing lower surface. The system further includes a layered Al delivery structure deposited on at least one of the upper surface and the lower surface. The system further includes a detection stage configured to receive the utensil and detect a usage indication signal from the utensil. The system includes one or more processors and one or more memories storing instructions that, when executed, cause the one or more processors to: determine a desired amount of the Al to be provided to the subject orally based on subject profile data; determine an initial amount of the Al on the utensil; determine, from the usage indication signal, an actual amount of the Al remaining on the utensil or an actual amount of the Al removed from the utensil; and determine desired modifications to the Al structure configuration on the utensil, or desired type of utensil, or prescribed dosing regimen, from a comparison of the actual amount of the Al remaining on the utensil or an actual amount of the Al removed from the utensil and the desired amount of the Al to be provided to the subject.

In another aspect, described herein is a utensil usage stage having a housing and a sample area for positioning at least the working end of an augmented utensil for inspection. The utensil usage stage includes a detection stage (also termed a station) positioned to detect a usage indication signal from the utensil; and a processing stage comprising at least one processor and at least one memory, the processing stage being configured to determine from the usage indication signal detected by the detection stage at least one of absence of the Al on the utensil, presence of the Al on the utensil, an amount of API absent from the utensil, an amount of Al present on the utensil. In some examples, the utensil usage stage comprises a housing; a sample area for positioning at least the working end of the utensil of claim 1 for inspection; a detection stage positioned to detect a usage indication signal from the utensil; and a processing stage comprising at least one processor and at least one memory, the processing stage being configured to determine from the usage indication signal detected by the detection stage at least one of absence of the Al on the utensil, presence of the Al on the utensil, an amount of Al absent from the utensil, an amount of Al present on the utensil. In an example, the utensil usage stage further comprises: an illumination activation stage for illuminating the utensil in the sample area with a detection illumination, wherein the detection stage comprises a photodetector for detecting the usage indication signal. In an example, the utensil usage stage further comprises: a thermal activation stage for applying a temperature change to the utensil in the sample area, wherein the detection stage comprises a thermal detector for detecting the usage indication signal. In an example, the utensil usage stage further comprises: a mount in the sample area configured to hold the elongated handled end and/or the working end.

In another aspect, described herein is an augmented utensil. The utensil includes an elongated handle end to be held in place during provision of an active pharmaceutical ingredient (Al) to a subject; a working end coupled to and extending from the handle end, the working end shaped to deliver the Al to the subject orally, the working end having an upper surface and an opposing lower surface; and a layered Al delivery structure deposited on at least one of the upper surface and the lower surface. In an example, the utensil comprises an elongated handle end to be held in place during provision of the API to the subject; and a working end coupled to and extending from the handle end, the working end shaped to deliver the Al to the subject orally, the working end having surface onto which, directly or indirectly, the Al, and optionally the excipient(s), is or coated or layered. In an example, the Al delivery structure comprising a solid Al film layer comprising greater than or equal to about 99 mass % of the Al as a deposited low molecular weight organic active ingredient compound having a molecular weight of less than or equal to about 1,000 g/mol.

In an example, the utensil is a container. In some examples, the utensil is a fork, spoon, blade, chopstick, cup, sippy cup, lid of cup, lid of sippy cup, or lid of a reusable water bottle. In an example, the utensil is a fork. In an example, the utensil is a spoon. In an example, the utensil is a blade. In an example, the utensil is a chopstick. In an example, the utensil is a cup. In an example, the utensil is a sippy cup. In an example, the utensil is a lid of cup. In an example, the utensil is a lid of sippy cup. In an example, the utensil is a lid of a reusable water bottle.

In some examples, the augmented utensil is patterned with one or more APIs at a working end to allow for oral administration to the subject. In some examples, the utensil is additionally patterned with an excipient agent that may also be a binding agent, a tracking agent, a kinetic agent, a flavor masking agent, a moisture responsive agent, a coloring agent, a temperature responsive agent, a delivery delay agent, a texture agent, a protective layer, an adhesion control agent, or any combination thereof.

In some examples, the augmented utensil contains a system of layered particles, such as a matrix, in which particles are embedded. The matrix may be separated from the utensil's surface by a sacrificial film. When the person is eating (a rice dish, soup, etc.), the coating is stable, save for the sacrificial film that permits the coating to slide off the utensil and into the mouth. Thus, the matrix (and/or particles themselves) may contain a taste-masking agent. Upon transit into the stomach, the matrix and outer layer of the particles dissolve. The remaining particles are surrounded by an acid-impervious (or a buffering) enteric layer, which protects the core of the particle from stomach acid and permits the particles to enter the intestine. This enteric coating dissolves in the intestine and exposes the internal core of the particle. The internal core of the particle may be 100% Al, or Al mixed with other ingredients, which may include excipients, buffers, co-formers, other Als, etc.]

In an example, the utensil comprises a layered Al delivery structure comprising the Al and at least one excipient layer for affecting kinetics of provision, and/or pharmacokinetics, and/or bioavailability, of the Al to the subject orally. In an example, the utensil further comprises a layered Al delivery structure comprising the Al and at least one excipient layer comprising a flavor masking layer film comprising a flavor masking agent for altering a perceived taste of the Al during provision of the Al to the subject orally. In an example, the utensil further comprises a layered Al delivery structure further comprising the Al and at least one excipient layer comprising a delivery delay layer film comprising a delaying agent for delaying absorption of the Al to the subject after provision of the Al to the subject orally. In an example, the utensil further comprises a layered Al delivery structure further comprising that Al and at least one excipient layer comprising a tracking ingredient separate from the Al, an amount of the tracking ingredient associated with the utensil in the second state, and thus after use by the subject, being indicative of an amount of the Al used by the subject.

In an example, the at least one excipient layer is adjacent to the Al film layer. In an example, the Al film layer is deposited on one of a first surface and a second, opposing surface of the utensil and the at least one excipient layer is deposited on the other of the first surface and the second surface of the utensil. In an example, the at least one excipient layer comprises at least one of a flavor masking agent, a delaying agent, a texturing agent, a moisture responsive agent, a coloring agent, a temperature responsive agent, a delivery delay layer film, adhesion control layer, and a protective layer. In an example, the layered Al delivery structure further comprises an indicator layer comprising a color indicator or symbology indicator, the indicator layer being at least partially covered by the Al film layer such that upon provision of the Al to the subject orally, the indicator layer becomes exposed or further exposed. In an example, the layered Al delivery structure further comprises a tracking layer that produces a signal when removed from the utensil or ingested by the subject. In an example, the layered Al delivery structure further comprises a tracking layer that in a first state of the utensil is prevented from detection by the Al delivery structure and that in a second state of the utensil corresponding to removal of at least a portion of the Al is detectable.

In an example, the utensil further comprises a layered Al delivery structure comprising a plurality of solid Al film layers, each solid Al film layer having a different Al. In an example, the utensil further comprises a layered Al delivery structure comprising a plurality of solid Al film layers, each solid Al film layer having a different cross-sectional thickness than at least one other solid Al film layers. In an example, the utensil further comprises a layered Al delivery structure comprising the Al and a non-Al material. In an example, the layered Al delivery structure comprises an Al film layer having the Al at patterned locations of the Al film layer and a non-Al material between the patterned locations. In an example, the Al is encapsulated in the Al film layer by a nano-capsule or micro-capsule at the patterned locations. In an example, the Al film layer comprises the Al encapsulated by one or more nano-capsules or micro-capsules.

In some examples, the utensil is a spoon, a fork, a blade, or a combination thereof. In some examples, the utensil is a container having a lid, the utensil further comprising a layered Al delivery structure layered or deposited on the lid of the container, such as a cup or sippy cup lid, or the cup itself, or the leading end of a container or utensil. In some examples, the utensil further comprises a layered Al delivery structure layered or deposited on a leading edge of the utensil.

In some examples, the utensil further comprises a layered Al delivery structure comprising small-molecule pharmaceutical ingredients, biologics, proteins, antibodies, excipients, gels, polymers, and/or inactive ingredients. In an example, the layered Al delivery structure comprises a three-dimensional (3D) printed biologic layered or deposited on the utensil. In an example, the utensil further comprises imprinted or labeled on the utensil Al identification data, subject identification data, drug regimen dosage identification data, and/or subject specific instruction data. In some examples, the Al is selected from the group consisting of an active pharmaceutical agent, a supplement, and a placebo.

Examples of Als include a compound selected from the group consisting of caffeine, acetaminophen (paracetamol), memantine, carbamazepine, 5-methoxy sulfadiazine, ethenzamide, nalidixic acid, isoniazid, furosemide, sulfadimidine, celecoxib, temozolamide, piroxicam, tryptamine, chlorzoxazone, p-coumaric, itraconazole, fluoxetine, telaprevir, sildenafil, theophylline, aceclofenac, 5-nitrouracil, indomethacin, aripiprazole, and atorvastatin, or a mixture thereof. Further Als may include by way of non-limiting example, various drugs or potential drugs (e.g., new chemical entities), including anti-proliferative agents; anti-rejection drugs; anti-thrombotic agents; anti-coagulants; antioxidants; free radical scavengers; nucleic acids; saccharides; sugars; nutrients; hormones; cytotoxins; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; acetylcholinesterase inhibitors; and combinations thereof. The description of these suitable organic compounds/pharmaceutical active ingredients/new chemical entities is merely exemplary and should not be considered as limiting as to the scope of compounds or active ingredients which can be applied to a surface according to the present disclosure, as all suitable organic molecules and/or active ingredients known to those of skill in the art for these various types of compositions are contemplated. Furthermore, an organic compound may have various functionalities and thus, can be listed in an exemplary class above; however, may be categorized in several different classes of active ingredients.

Examples of supplements include but are not limited to: ergocalciferol, cholecalciferol (Vitamin D3), vitamin D4, vitamin D5, ascorbic acid (vitamin C), retinol, vitamin B compounds, alpha tocopherol, menadione, creatine.

In some examples, a detection station is used to analyze the utensil to determine Al usage. For example, the detection station may have a sample area for examining an amount of Al remaining on the utensil after use and automatically communicating that amount to a mediation adherence system that determines medication protocol adherence. In some examples, the detection station is a chemical compound composition detection system that examines bodily fluid or bodily solids taken from the subject and determines the presence of a tracking agent indicating delivery of the Al. In some examples, the detection station detects an amount of the tracking agent to determine medication adherence. In some examples, the detection station detects the presence of the tracking agent, without detecting an amount, to determine medication adherence.

In some examples, a detection station may be configured to examine a package of utensils to determine how many of the utensils are present or not present in the package and/or to determine an amount of Al remaining on utensils in the package and/or an amount of Al removing from utensils in the package.

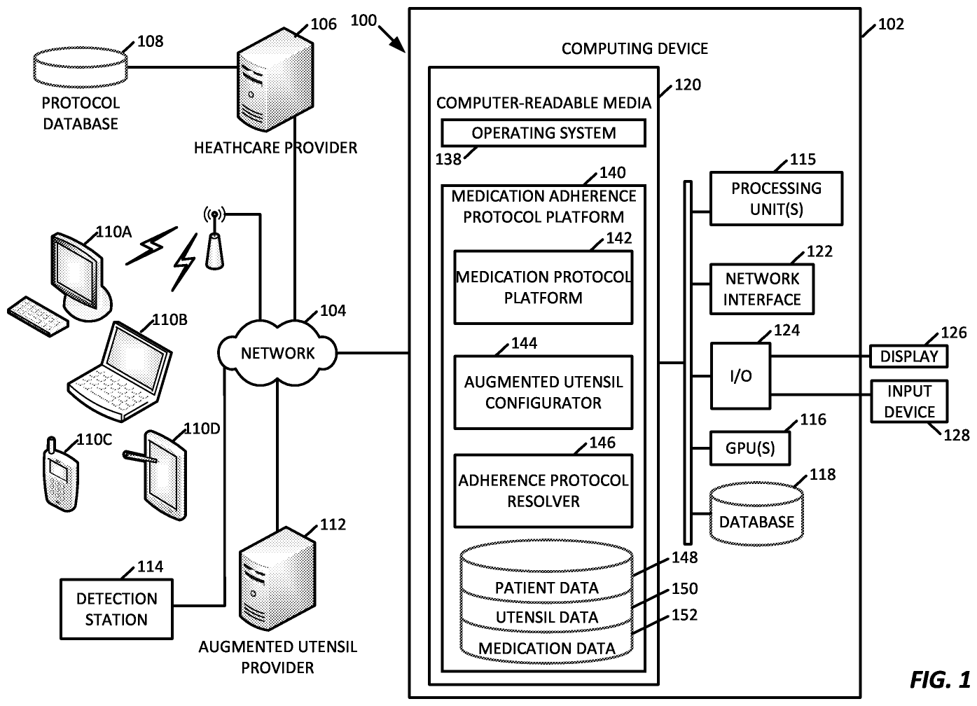

FIG. 1 illustrates a medication protocol adherence system 100, in an example. A computing device 102 of the system 100 for implementing the processes described and illustrated herein is coupled to a network 104 for receiving and communicating various types of data. The network 104 may be a wireless or wired network communicating with various computing devices, including, in the illustrated example, a healthcare provider computing system 106 having a medication protocol database. The healthcare provider computing system 106 may represent a physician's computing terminal at a hospital or other healthcare facility. The protocol database 108 may store medication data for a plurality of active or inactive ingredients (such as active ingredients such as Als or supplements) to be used in treating a patient, in particular active ingredients available for oral delivery to a patient. The protocol database 108 may store data including, but not limited to, optionally: molecular weight of an Al, chemical structure, chemical database reference number(s), solubility, enthalpy of vaporization, vapor pressure, melting point, thermal decomposition point, viscosity, hardness, taste profile, counterindications, co-prescriptions, dosage, expiration dating information, storage instructions, safety instructions, associated billing codes, prescribed regimen for a given patient, regimen starting time and date, regimen ending time and date, patient gender, body weight, height, body mass index, genetic information, place of administration, manner of administration, administration restrictions (e.g. temperature sensitive Als restricted from certain utensils), preferred foods, counter-indicated foods, person responsible for administration, patient address, service subscription information, service renewal information, etc. As discussed in examples herein, a healthcare professional may provide medication protocol data to the provider system 106 for providing the medication protocol data to the computing device 102.

In the example of FIG. 1, various patient devices are also connected to the network 104 through which patients can enter identification data to the computing device 102. The patient devices may present an instantiation of an accessing (app) to provide such identification data and to allow the patient devices to be individually authenticated from communication with the computing device 102. In the illustrated examples, patient devices include a computing terminal 110A, a laptop computer 110B, a mobile cellular device 110C, and a mobile tablet computing device 110D, each available to a different patient. Other patient devices include personal mobile and/or monitoring devices, such as smart watches and other wearable monitoring devices such as heart rate monitors and heath things. Of course, provided devices may be coupled to the computing device 102 through the network 104 and these provided devices may be computer terminals, laptop computers, mobile cellular devices, mobile tables, and the like, as well.

Further, as shown, an augmented utensil provider 112 and a detection station 114 are connected to the computing device 102 through the network 104. The augmented utensil provider 112, as discussed herein, may receive augmented utensil configuration data from the computing device 102 and generate augmented utensils having an API (or “active ingredient”) layered structure formed and patterned to provide medication to a patient and to allow for automated tracking of the amount of medication consumed by the patient, for example, using the detection station 114. The augmented utensil provider 112 may represent a computing and fabrication system configured in various different ways to fabricate augmented utensils. Example types of computing and fabrication systems include but are not limited to, systems configured for dip-coating, vacuum coating, spray-coating, ink-jet printing, organic vapor jet printing, chemical vapor deposition, organic vapor phase deposition, sputtering, dip-pen coating, soaking, 3D printing (of inorganic, organic, and/or biological materials) to deposit an active ingredient (e.g., an inorganic or organic compound).

In the example of FIG. 1, the medication protocol adherence system 100 is implemented on a single network accessible server. In other examples, the functions of the medication protocol adherence system 100 may be implemented across distributed devices connected to one another through a communication link. In other examples, functionality of the medication protocol adherence system 100 may be distributed across any number of devices, including the portable personal computer, smart phone, electronic document, tablet, desktop personal computer devices shown, and personal mobile and/or monitoring devices, such as wearable devices like smart watches, health rings, and heart rate monitors. In other examples, the functions of the medication protocol adherence system 100 may be cloud-based, such as, for example one or more connected cloud CPU (s) customized to perform machine learning processes and computational techniques herein.

The network 104 may be a public network such as the Internet, private network such as research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), Bluetooth, Bluetooth Low Energy, AirPlay, or other types of protocols. Moreover, the network 104 can include a number of devices that facilitate network communications and/or form a hardware basis for the networks, such as switches, routers, gateways, access points (such as a wireless access point as shown), firewalls, base stations, repeaters, backbone devices, etc.

The computing device 102 includes one or more processing units 115, one or more optional graphics processing units 116, a local database 118, a computer-readable memory 120, a network interface 122, and Input/Output (I/O) interfaces 124 connecting the computing device 102 to a display 126 and user input device 128.

The computer-readable media 120 may include executable computer-readable code stored thereon for programming a computer (e.g., comprising a processor(s) and GPU(s)) to the techniques herein. Examples of such computer-readable storage media include a hard disk, a solid state storage device/media, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 102 may represent a CPU-type processing unit, a GPU-type processing unit, a field-programmable gate array (FPGA), another class of digital signal processor (DSP), or other hardware logic components that can be driven by a CPU.

In the illustrated example, in addition to storing operating system 138, the memory 120 stores a medication adherence protocol platform 140, configured to execute various processes described and illustrated herein. In an example, the drug-target optimization platform 140 includes a medication protocol platform 142 and an augmented utensil configurator 144, each in accordance with example techniques described herein. Additionally, the memory 120 includes an adherence protocol resolver 146 and plurality of different databases, in this example, a patient data database 148, a utensil data database 150, and a medication data database 152.

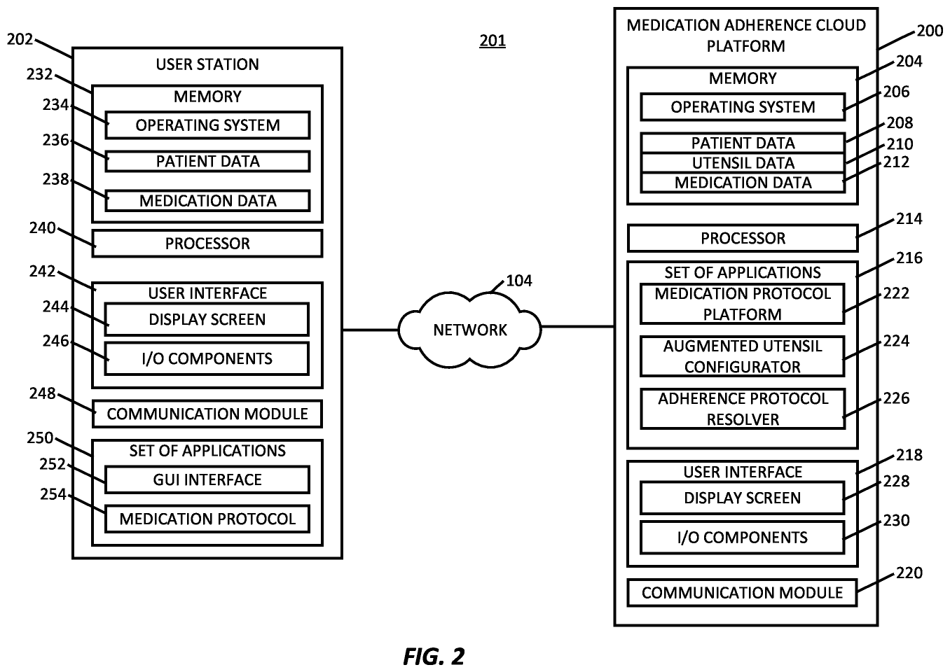

FIG. 2 illustrates an implementation of the medication protocol adherence system 100 with a medication adherence cloud platform 200 and example user station 202, in this example a patient station, in a first cloud-based configuration 201. In some examples, the user device 202 may perform the functionalities as discussed herein as part of a “cloud” network (e.g., via network(s) 104 and the medication adherence cloud platform 200) or may otherwise communicate with other hardware devices or software components within the cloud to send, retrieve, or otherwise analyze data. In some embodiments, the medication adherence cloud platform 200 may operate as a Software-as-a-Service (SaaS) or Platform-as-a-Service (PaaS), providing the functionality of the medication adherence cloud platform 200 remotely to software apps and other components in accordance with the various embodiments described herein.

The cloud platform 200 includes computer readable memory 204 having an operating system 206 and storing patient data 208, utensil data 210, and medication data 212. The cloud platform 200 further includes processor 214, a set of applications (apps) 216, (optionally) a user interface 218, and a communication module 220 for communication over the network 104.

The set of applications 216 include a medication protocol platform 222, an augmented utensil configurator 224, and an adherence protocol resolver 226. In accordance with processes described and illustrated herein, in some examples, the medication protocol platform (app) 222 is configured to receive and/or generate a medication adherence protocol in response to patient identification data received from the user station 202. In some examples, the medication protocol platform 222 accesses the patient data 208 and the medication data 212 and performs a comparison to determine a medication adherence protocol and includes one or more Als to be administered to a patient. In accordance with processes described and illustrated herein, in some examples, the augmented utensil configurator (app) 224 accesses the utensil data 210 and the medication adherence protocol from the platform 222 and develops an augmented utensil configuration data that includes Al (or “active ingredient”) layered structure data that includes the Als and excipient agents and a layer structure thereof for application to a utensil to form an augmented utensil. The augmented utensil configuration data may further include pattern data defining a location and pattern of the Al layered structure on the utensil to form the augmented utensil. In some examples, the augmented utensil configuration data may be packaged as instructions to be sent to augmented utensil provider (such as 112 in FIG. 1) for use in fabricating an augmented utensil or a plurality of utensils in accordance with the augmented utensil configuration data. In some examples, the augmented utensil configuration data includes data on already fabricated augmented utensils.

In accordance with processes described and illustrated herein, in some examples, the adherence protocol resolver (app) 226 is configured to receive usage data for the user station 202 or from a detection station (such as 114 in FIG. 1) and compare the usage data to the medication adherence protocol of platform 222 and determine from such comparison a predicated medication adherence of the patient.

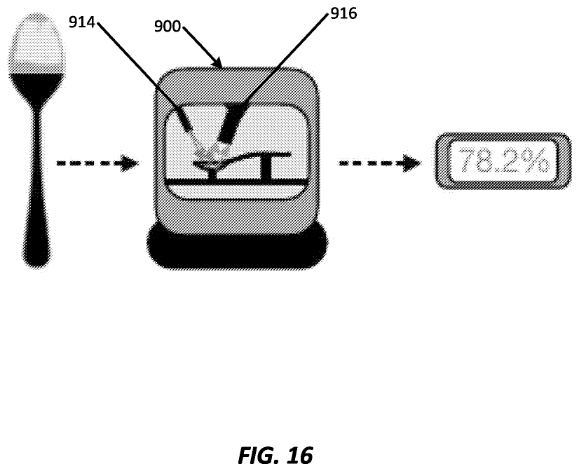

In this way, the set of apps 216, which may include GUIs for allowing user access and data entry and authorization, is capable of performing tracking of patient medication protocol adherence. Thus, the medication adherence cloud platform 200 is a computing device able to the receive identification data on a subject, including a pathology data for the subject and determine from the identification data a desired drug regimen in form for the medication adherence protocol. That protocol may be then be converted, by the medication adherence cloud platform 200, into augmented utensil configuration data that is specific to the subject and specific to the type(s) of utensils to be augmented. That augmented utensil configuration data may include (i) a desired amount of one or more Als to be administered to the subject for treating a condition using an augmented utensil having a working end having layered with an Al delivery structure formed thereon. That augmented utensil configuration data may further include, in some examples, at least one parameter of the augmented utensil, such as shape, comprising material, intended packaging, utensil vendor, batch number, storage requirements, serial number, patient data, regimen-related data, compatible foods, etc. Further, the medication adherence cloud platform 200 can receive from a detection station (not shown, examples include station 114 in FIG. 1 and station 900 in FIG. 16) the amount of used Al and compare the amount of used Al to the desired amount of the Al stored in the augmented utensil configuration data and determined from the medication adherence protocol. From the comparison of the amount of used Al to the desired amount of the Al, the medication adherence cloud platform 200 is able determine an adherence score indicating a predicted adherence of the subject to the medication protocol.

The user interface 218 of the medication adherence cloud platform 200 includes (optionally) a display screen 228 that may be used to display a GUI with screens generated by the apps 216 for user station authentication, detection station authentication, and data review and entry related to the processes of the apps 216. Further display screen 228 may be used to display a report of the predicted adherence score for examination by a user, such as a healthcare professional. The user interface 218 may include I/O components for allowing a user to interface with the cloud platform 200 through external interfacing devices, e.g., computers, handheld devices, user terminals, wearable devices, and the like.

In the example of FIG. 1, the user station 202 includes a computer readable memory 232 including an operating system 234 and patient data 236 such as identification data that includes authentication data, demographic data, medical conditions data, medical history data, medication adherence historical data, and/or preferred taste profile(s), counterindications, co-prescriptions, dosage, expiration dating information, storage instructions, safety instructions, associated billing codes, prescribed regimen for a given patient, regimen starting time and date, regimen ending time and date, patient gender, body weight, height, body mass index, genetic information, place of administration, manner of administration, administration restrictions (e.g. temperature sensitive APIs restricted from certain utensils), preferred foods, counter-indicated foods, person responsible for administration, patient address, service subscription information, service renewal information, etc. In some examples, the memory 232 includes medication data 238 such as prescribed medication, prescribed medication protocols, and/or preferred taste profile(s), counterindications, co-prescriptions, dosage, expiration dating information, storage instructions, safety instructions, associated billing codes, prescribed regimen for a given patient, regimen starting time and date, regimen ending time and date, patient gender, body weight, height, body mass index, genetic information, place of administration, manner of administration, administration restrictions (e.g. temperature sensitive Als restricted from certain utensils), preferred foods, counter-indicated foods, person responsible for administration, patient address, service subscription information, service renewal information, etc.

The user station 202 further includes a processor 240, and user interface 242 having a display screen 244 and I/O components 246. The user station 202 further includes a communication module 248 for communicating with the medication adherence cloud platform 200 over the network 104. A set of applications (apps) 250 includes a GUI interface (app) 252 for interaction by a user and a medication protocol app 254 for the user to be provided with medication protocol data, both via the display screen 244. In some examples, the user station 202 is implemented as a network-accessible computing device, server, or other device that is accessed by the cloud platform 200 without specific user input. Such configurations may be particularly useful for patients incapable of providing user input to a device, such as infirmed patients. In some such examples, the user station 202 is essentially “dark” to the user, and the data in memory 232 is accessed by the cloud platform 200 in an automated manner to access the patient data 236 and the medication data 238. In such examples, the patient or caretaker may simply use the utensils in accordance to protocols and utensils configured in response to and monitored by the cloud platform 200.

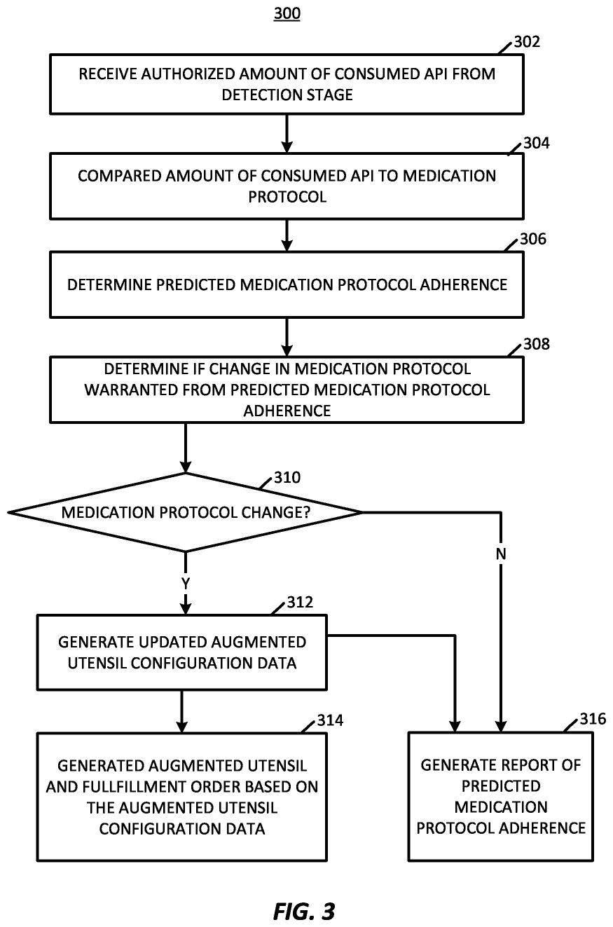

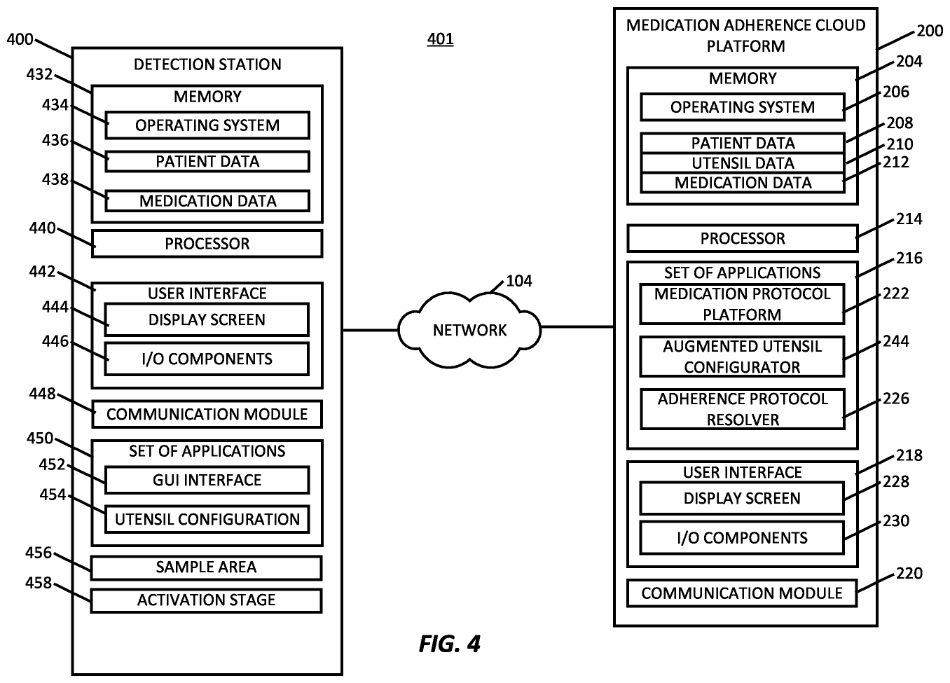

FIG. 3 illustrates an example process 300 for predicting medication protocol adherence as may be performed by the medication protocol adherence system 100 and/or the medication adherence cloud platform 200. At a process 302, a medication protocol adherence system receives an authorized amount of consumed API from a detection stage, such as detection station 114 or detection station 400 (FIG. 4). The amount of consumed API may include an amount of multiple, different consumed API's, if an augmented utensil included multiple APIs in its Al layered structure. In some examples, the process 302 receives an amount of consumed Al that the detection station has determined by first detecting an amount of a target agent and from the detected amount of target agent determined an amount of consumed Al. In some examples, the process 302 receives a detected amount of the target agent, from which the process 302 determines an amount of consumed Al. To authenticate the amount of consumed Al, in some examples, the medication protocol adherence system authorizes the detection station before receiving such Al and/or target agent consumption. Authentication may include authenticating the detection station as authorized to detect an augmented utensil. For example, only certain detection stations or certain types of detection stations may be authorized by the medication protocol adherence system to detect Al usage on particular augmented utensils. Indeed, in some examples, such coordination may be achieved by assigning detection stations to detect only those augmented utensils associated with a particular identification number, usage pattern, time of day, location, and/or other parameters.

At a process 304, the medication adherence cloud platform 200 accesses medication protocol data (e.g., stored at the data 212) identifying an amount of one or more Als to be taken, a medication delivery time frame for each of the one or more Als, preferred taste profile(s), counterindications, co-prescriptions, dosage, expiration dating information, storage instructions, safety instructions, prescribed regimen, regimen starting time and date, regimen ending time and date, patient gender, body weight, height, body mass index, place of administration, manner of administration, administration restrictions (e.g. temperature sensitive Als restricted from certain utensils), preferred foods, counter-indicated foods, person responsible for administration, patient address, service subscription information, service renewal information, etc. The process 304 compares the received authorized amount of consumed Als to the medication protocol data and determines the difference in amount of Al consumed against the amount of Al prescribed in the medication protocol. A process 306 determines a predicted medication protocol adherence, for example, whether the amount of consumed Al is less than the amount of prescribed Al. The process 306 may apply an Al specific rule in making the determination. Some Als, for example, may correspond to medications that allow a percentage deviation in the amount of medication consumed versus the amount of medication prescribed. Other Als may correspond to medications that require stricter adherence and do not allow for a percentage deviation in the amount of medication consumed. The process 306 accessing the medication data 212 applies such Al specific rules and predicts adherence to the medication protocol. That prediction may be a binary scoring, of “ON” protocol or “OFF” protocol, while in other examples, the prediction may be a scored value over a range. The comparison is a prediction in that, while the present techniques provide for highly accurate assessment of Al delivered, the comparison still presumes that the intended subject actually consumed the Al using the augmented utensil.

From the determined predicted medication protocol adherence, a process 308 determines if a change in medication protocol is warranted. For example, the process 308 may access medication protocol data and automatically determine, in the presence of a failure to adhere to the medication protocol, that an increase in one or more of the Als is warranted. If a medication protocol change is warranted, a process 310 passes control to a block 312 that generates updated augmented utensil configuration data, having new Al levels, stored as a new Al layered structure data, that is to be used in forming a new augmented utensil. The medication adherence cloud platform 200 may then transmit the updated augmented utensil configuration data to an augmented utensil provider for fabrication of augmented utensils according to the updated configuration data, at a process 314. The process 312 also sends the predicted medication protocol adherence score to a process 316 were a report is generated for display to a user accessing the medication adherence cloud platform 200. The report may be presented to the user station 202, a healthcare provider computing system, or other system accessing the platform 200.

FIG. 4 illustrates the medication adherence cloud platform 200 communicatively coupled to augmented utensil fulfillment station 500 through the network 104 in a second cloud based configuration 401. The detection station 400 includes a computer readable memory 432 including an operating system 434 and patient data 436 received from the cloud platform 200. In some examples, the memory 432 includes medication data 438 also received from the platform 200. The detection station 400 further includes a processor 440, and user interface 442 having a display screen 444 and 1/O components 446. The detection station 400 further includes a communication module 448 for communicating with the medication adherence cloud platform 200 over the network 104. A set of applications (apps) 450 includes (optionally) a GUI interface (app) 452 for interaction by a user and a utensil configuration app 454 in which utensil configuration data is obtained and modified, e.g., by a user, to be used in assessing the amount of API consumed as indicated by the examining for a target agent of the augmented utensil. The detection station 400 further includes a sample area 456 onto which a utensil is mounted such that a working end of the utensil can be examined for consumed Al or for presence of a target agent. An activation stage 458, such as an illumination and/or imaging stage, is used to examine the utensil at the sample area 456 for making this assessment.

In some examples, the detection station 400 station may include a microscope or spectroscope. In some examples, the detection station 400 can observe a color change in an indicator dye present on the augmented utensil, and which dye has been introduced to the augmented utensil during manufacturing. In some examples, the detection station 400 can use an indicator or sacrificial substrate that characterizes its contents via an integrated mechanism (e.g., through built-in color filters), without requiring a separate microscope or spectroscope.

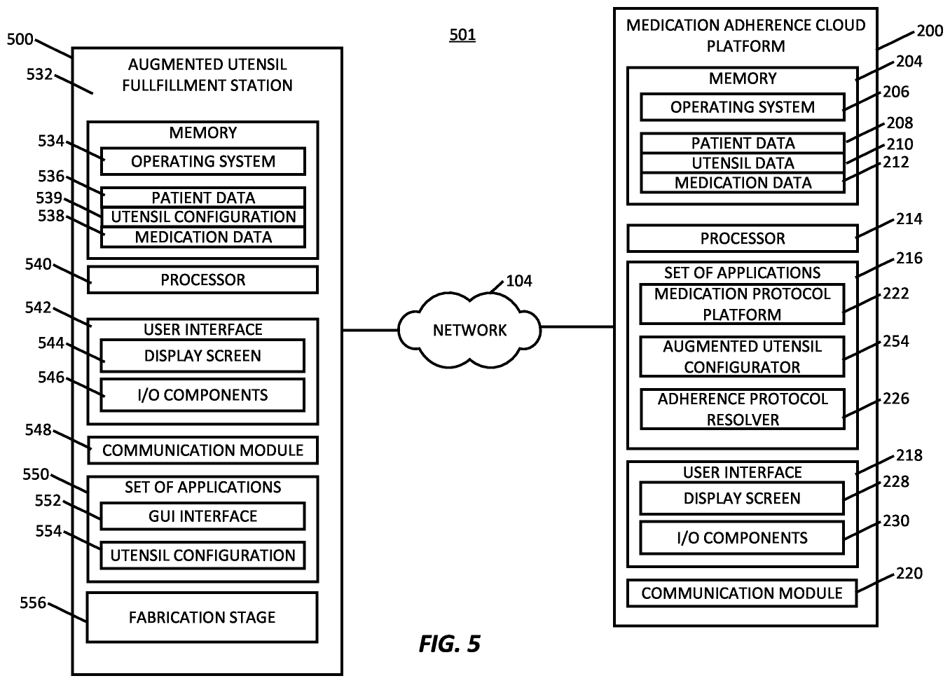

FIG. 5 illustrates the medication adherence cloud platform 200 communicatively coupled to an augmented utensil fulfillment station 500 for augmented utensils and/or packaging augmented utensils during manufacturing, including, in some examples through fully or partially automated ways under control of the cloud platform 200. In the illustrated example the fulfillment station 500 is connected to the cloud platform 200 through the network 104, in a third cloud-based configuration 501. The augmented utensil fulfillment station 500 includes a computer readable memory 532 including an operating system 534 and patient data 536 received from the cloud platform 200. In some examples, the memory 532 includes medication data 538 also received from the platform 200 and augmented utensil configuration data 539 similarly received from the platform 200. The augmented utensil fulfillment station 500 further includes a processor 540, and user interface 542 having a display screen 544 and 1/O components 546. The augmented utensil fulfillment station 500 further includes a communication module 548 for communicating with the medication adherence cloud platform 200 over the network 104. A set of applications (apps) 550 includes a GUI interface (app) 552 for interaction by a user and a utensil configuration app 554 in which utensil configuration data is obtained and modified, e.g., by a user, to be used in assessing the amount of Al consumed as indicated by the examining for a target agent of the augmented utensil. The augmented utensil fulfillment station 500 further includes a fabrication stage 556 at which augmented utensils are formed in accordance with the utensil configuration data. Example fabrication techniques and fabrication stages are discussed in further examples herein.

In some examples, the computing device 102, the medication adherence cloud platform 200, and/or the augmented utensil fulfillment station 500 may include an analysis stage, such as computer-assisted analysis of experimental data. That analysis stage may be configured to include combinations of test components based on computerized analysis of an initial test or screen of a single or combination of pharmaceutical agents, supplements, and excipient agents. In some examples, the analysis stage performs integrated data analysis that allows for automated decision-making and further analysis of a particular subset of initially-tested combinations of components or variables. The analysis stage can be partially automated, or fully automated.

In some examples, the analysis stage is implemented in part by a control module. For example, the control module may receive data from the analysis stage such as spectroscopic data from a microscopy/spectroscopy module providing response data from applying different drugs to different compounds. Based on the microscopy/spectroscopy response data, the control module may modify combinations of active ingredients and or modify of patterns of active ingredients to be formed as solid films on working end of a utensil. In some examples, the control module may be determine that different drug combinations should be generated by the vapor deposition device for testing of targets in the substrate/assay plate. The control module may be adjusted in response to the microscopy/spectroscopy response data.

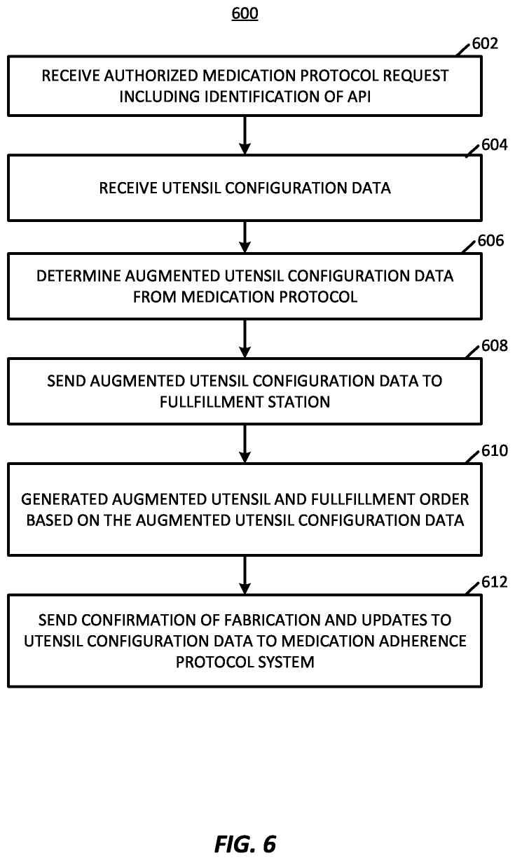

FIG. 6 illustrates a process 600 for generating an augmented utensil using the cloud-based configuration 501, in an example. At a process 602, the medication adherence cloud platform 200 receives authorized medication protocol request from a healthcare provider station. In some examples, that protocol request includes identification data for one or more Als to be administered to the patient using an augmented utensil. At a process 604, the medication adherence cloud platform 200 receives utensil data corresponding to the utensil to be augmented. This augmented utensil data may include data identifying the shape of the utensil, the shape and/or configuration of a working end of the utensil, material properties of the utensil, existing coatings on the utensil, the location of an API on a working end of the utensil, etc. In some examples, the augmented utensil data includes data identifying one or more of an Al layered structure data of the utensil, one or more excipient layers of the utensil, a flavor masking layer of the utensil, a delivery delay layer of the utensil, a tracking ingredient of the utensil, a non-API material of utensil, and/or a pattern of locations of the Al on the utensil. At a process 606, the medication adherence cloud platform 200 applies the data from processes 602 and 604 to a conversion module containing models of data transformations that assign different combinations of Al identifiers and utensil configuration data to different Al layered structures containing one or more layers of Als and one or more excipient agents/layers as well as patterns for where to form the Al layered structures on a utensil working end. The augmented utensil configuration data of process 606 is communicated over the network 104 to the augmented utensil fulfillment station 500 at process 608, and the augmented utensils are fabricated by the station 500 at process 610, after which the station 500 may send confirmation data back to the platform 200 at process 612. At the process 612, for example, the station 500 may send identification data associated with the augmented utensils, such as barcode data or other identifying indicia, to the medication adherence cloud platform 200 for associating the specific fabricated utensils with the specific corresponding medication protocol data. This will allow the medication adherence cloud platform 200 to later authenticate subsequent detected consumed Al data.

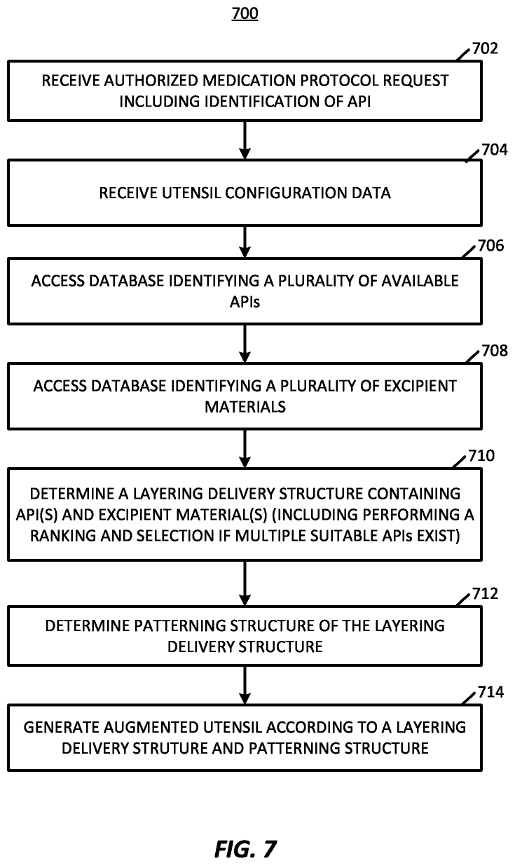

FIG. 7 illustrates an example detailed process 700 for generating an augmented utensil. The authorized medication protocol request is received at process 702 and utensil configuration data is received at process 704, similar to that of process 600. At a process 706, the medication adherence cloud platform 200 accesses a database identifying a plurality of available Als. This may be done by a prescribing physician, with consultation with patient and insurance company, with the aid of an electronic medical records service/database and formulary. At a process 708, the medication adherence cloud platform 200 access a database identifying a plurality of available excipient layers. At a process 710, the medication adherence cloud platform 200 determines a layering structure containing the identified APIs and excipient layers. Further, in some examples, multiple API's may be available that would satisfy a particular medication protocol. Therefore, in some examples the process 710 performs a ranking of all suitable Als and selects the Als to be applied to a utensil according to properties relevant to the method of manufacture. For example, if the manufacturing method includes soaking the utensil in a hydrogel solution, then Al solubility properties are prioritized over melting properties; if the manufacturing method includes dip-coating the utensil in a polymer melt, then the melting properties of the polymer Al or supplement are prioritized; if the manufacturing method includes organic vapor jetting of a small molecular Al or supplement, then the vapor pressure of the ingredient is prioritized over other properties.

At a process 712, the patterning structure of the layer delivery structure from process 710 is determined, and at a process 714 the patterning structure and layer delivery structure are used by a fulfillment station (such as station 500) to generate augmented utensils. That is, in this example, the medication adherence cloud platform 200 communicates the structure data to the fulfillment station for fabrication.

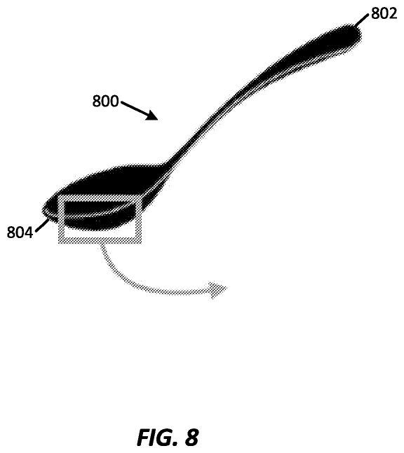

FIGS. 8-11 illustrate different examples of augmented utensils that may be formed in accordance with processes described and illustrated herein. In an example, a utensil 800 includes an elongated handle end 802 and a working end 804, as shown in the example of FIG. 8. The working end 804 is coupled to the elongated handle end 802 extending therefrom and shaped to deliver an Al to a subject orally. The working end may be shaped to form a spoon, a fork, a blade, or a combination thereof.

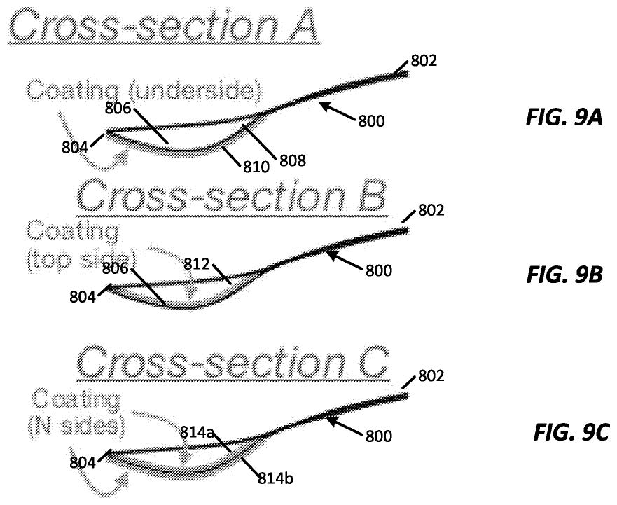

As shown in FIGS. 9A-9C, the working end includes an inner surface 806 (also termed an upper surface) and an outer surface 808 (also termed a lower surface). In FIG. 9A, an Al layered structure 810 is formed on the outer surface 808 and contains one or more Als corresponding to a medication protocol and as determined by a medication adherence platform system. This configuration represents a first pattern type, with the Al(s) formed on an outer surface of the working end. FIG. 9B illustrates another configuration, with an Al layered structure 812 formed on the inner surface 806. FIG. 9C illustrates yet another pattern with an Al layered structure 814 that as Al layers formed on both the outer surface 808 and the inner surface 806. As noted above, references herein to an Al or Al layered structure are meant to include active ingredients or active ingredient layered structures, respectively. That is, while Al is discussed, the structures could be those formed of any suitable active or inactive ingredient, whether pharmaceutical, supplement, taste-modifier, color indicator, tracer, or some combination thereof.

The Al layered structures (also termed layered Al delivery structures) may be formed over the entire outer surface 808 or the entire inner surface 806 or patterned over only portions thereof. For example, the layered Al delivery structure may be deposited on a leading tip portion of the upper surface or a leading tip portion of the lower surface, so that the medication is located at the leading end of the utensil 800 to be provided to the subject orally.

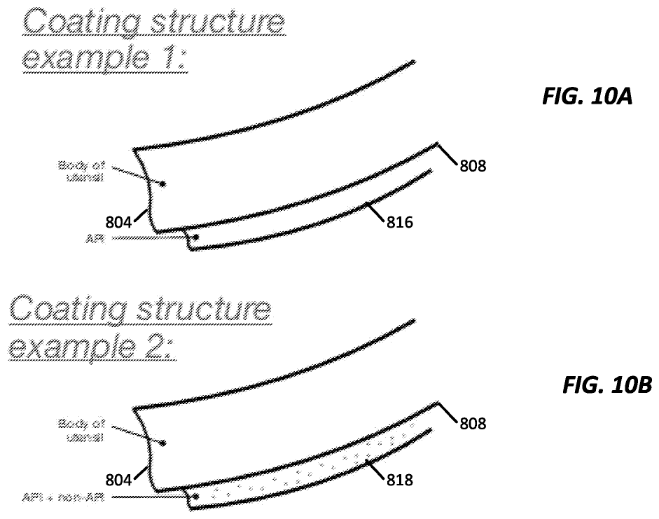

FIGS. 10A and 10B illustrate a cross section of a portion of the working end 804 with an Al layered structure formed thereon. In FIG. 10A, an Al layered structure 816 is formed of only one or more Als. In FIG. 10B, an Al layered structure 818 contains one or more Als and a non-Al in the form of an excipient agent within the structure. The excipient agent may be formed as a matrix within the layered structure 818, while in other examples, the excipient agent may be formed in a dedicated excipient layer with no Al, such that the excipient layer and Al layer form a multi-layered structure.

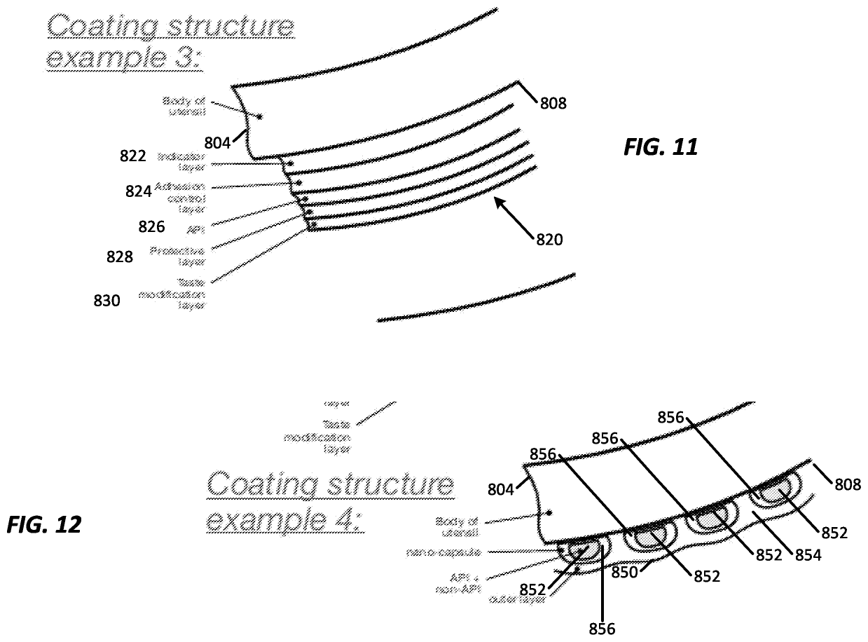

FIG. 11 illustrates a multi-layered Al delivery structure 820 having an indicator layer 822, an adhesion control layer 824, an Al layer 826, a protective layer 828, and a taste modification layer 830, in an example.

As shown in the example of FIG. 11, the excipient agents herein may be chosen for affecting kinetics of the provision of the Al to the subject orally. In some examples, the excipient agent is a flavor masking agent for altering a perceived taste of the Al during provision of the Al to the subject orally, including but not limited to: methacrylic acid, methacrylic ester copolymers, aspartame, acesulfame potassium, sucralose, lemon flavor, menthol flavor, banana flavor, peppermint flavor, citric acid, sodium cyclamate, zinc sulfate. In some examples, the excipient agent is a delivery delay agent for delaying absorption of the Al to the subject after provision of the Al to the subject orally, including but not limited to polylactic acid (PLA), poly(lactic-co-glycolic acid) (PLGA), Polyethylene glycol (PEG), silk fibroin, their derivatives, and others. In some examples, the excipient agent is a tracking ingredient separate from the Al, such that an amount of the tracking ingredient associated with the utensil after use by the subject is indicative of an amount of the Al used by the subject. In some examples, the tracking ingredient is barium fluoride and other radiopharmaceuticals, Fluorine-18, fluorescent dyes (ICG, IRDye8, Cy5.5, and many others as in 10.3389/fphar.2019.00510), lactulose, rhamnose, riboflavin, or MB-402/MB-301 detectable in a bodily fluid of the subject. In some examples, the tracking ingredient is a metabolite of one or more compounds ingested, detectable by an external means, such as spectroscopy (examples are described extensively in https://doi.org/10.1038/s41746-019-0185-y).

Thus, more generally, an excipient agent may include flavor masking agent, a delaying agent, a texturing agent, a moisture responsive agent, a coloring agent, a temperature responsive agent, a delivery delay layer film, adhesion control layer, a protective layer, a tamper indicator, contamination indicator, pH sensing/indicating agent, and/or an enteric coating.

As additionally shown in the example of FIG. 11, the layered Al delivery structure 820 may include the indicator layer 822 which may be a color indicator or symbology indicator, the indicator layer being at least partially covered by the Al film layer such that upon provision of the Al to the subject orally, the indicator layer becomes exposed or further exposed. In some examples, the symbology indicator is a barcode, QR code, speckle pattern, grid, or other decodable indicia.

The Al layer 826 may have a uniform cross-sectional thickness in some examples, while the cross-sectional thickness may vary in other examples. Further still, as shown in an example in FIG. 12, an Al layered delivery structure 850 may have an Al layer that contains an Al at patterned locations 852 and where a non-Al material 854 extends between the patterned locations 852. Further the Al at the patterned locations 852 may be encapsulated by a micro- or nano-capsule 856 at the patterned locations.

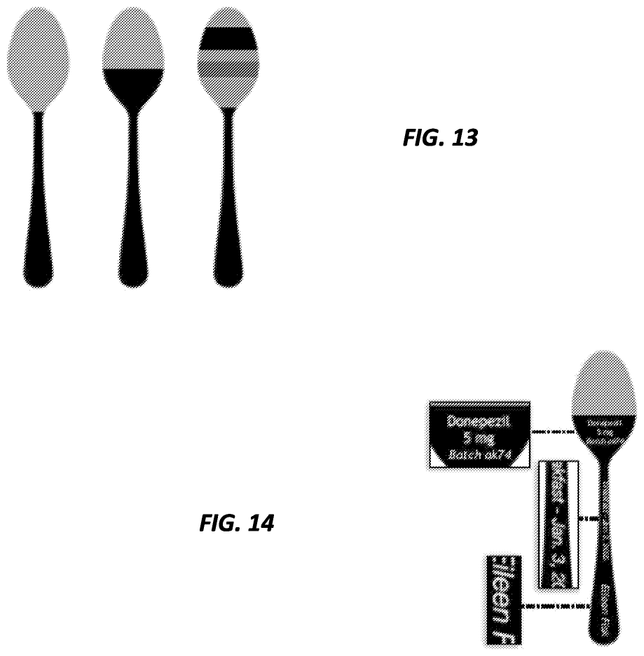

FIG. 13 illustrates three different augmented utensil configuration patterns, a first where the Al delivery structure covers the entire working end, a second where the Al delivery structure coves a leading portion of the working end, and a third where different layers forming the Al delivery structure are formed at different locations of the working end. FIG. 14 illustrates an augmented utensil showing an indicator layers with a text descriptor of the Al. The indicator layer may be visible at the time of fabrication. In other examples, the indicator layer may be fully or partially covered by an Al layer such that the indicator layer is partially or fully exposed only after some portion of the Al has been consumed by the subject. The indicator layer may indicate a subject name, a fabrication date, or date at which to take the medication or other indicator.

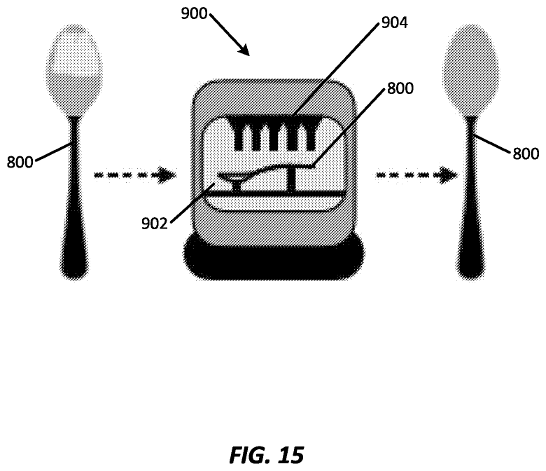

FIGS. 15 and 16 illustrate an example detection station 900 for detecting an amount of Al consumed on the utensil 800. The station 900 includes a sample area 902 configured to hold the utensil 800, for example, using a mount in the sample area configured to hold the elongated handled end and/or the working end. An activation stage 904 is positioned above the sample area 902 to examine the utensil and detect an amount of Al removed from and/or remaining on the utensil 800. In an example, the activation stage 902 is an illumination activation stage for illuminating the utensil in the sample area with a detection illumination. The activation stage may include a photodetector for detecting reflected illumination and generate a usage indication signal. In another example, the activation stage 902 is a thermal activation stage for applying a temperature increase in the sample area and there the detection station 900 includes a thermal detector for detecting the usage indication signal. FIG. 16 illustrates an example where the activation stage comprises two parts an activator 914 and a detector 916, in accordance with these examples.

In some examples, the detection station 900 is an imaging station having one or more cameras with a corresponding field of view that includes the sample area and captures images of the sample area, in particular images of augmented utensils or groups of augmented utensils. The captured images may be analyzed, by a computing device of the detection station, to determine an amount of Al consumed, remaining, or a combination thereof. In some examples, the computing device may use a pattern recognition module to assess the captures images to determine an amount of Al consumed, remaining, etc. In some examples, that pattern recognition module may be configured with trained classifiers developed using a machine learning framework, such as classifiers configured as a convolutional neural network.

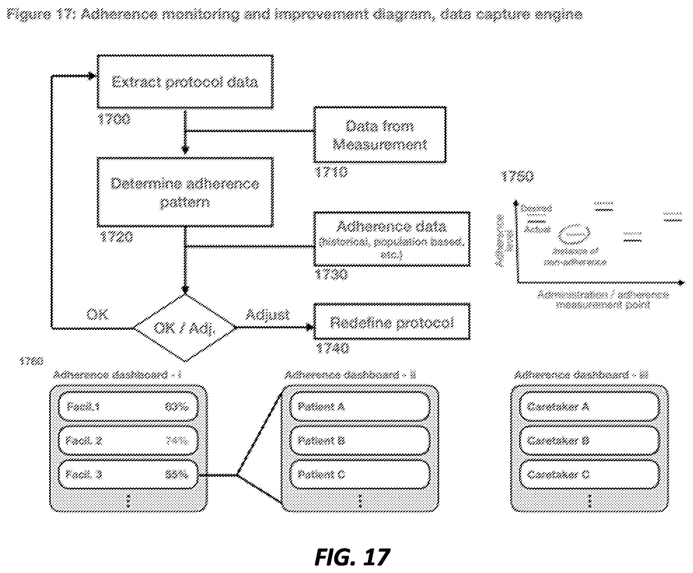

FIG. 17 illustrates an example process in accordance with techniques herein, in particular an example adherence monitoring and improvement process, as may be performed by a data capture engine implemented by an example system herein, such as the computing device 102 or the medication adherence cloud platform 200. At a process 1700, the system extracts data (1710) relating to, in various implementations, measurement of API remaining on the utensil, measurement of a biological fluid sample from the patient, measurement from a user device such as a phone or heart rate monitor or blood pressure monitor, etc. In some implementations, the data 1710 is collected as it is generated, including by remote monitoring systems/devices. In some implementations, data 1710 is batched and collected. Data 1710 may bear association with the patient, protocol, and utensil. In some examples, a detection station obtains and communicates the data 1710 to a medication adherence cloud platform, such as those shown in FIG. 4. In some examples, a cloud platform may pole a detection station for such data. In other examples, the data is collected and communicated by the detection station, such as in real-time or periodically. In some examples, the measurement data 1710 is dependent upon data on the protocol, the user, and/or the utensil. For example, a detection station may receive protocol data, user data, and/or utensil data and adjust detection parameters to more accurately detect for active ingredient usage based on that data. For example, data may be received at the detection station of the type of tracking agent or tracking layer or tracking insignia used in a utensil and use that data to adjust detection parameters for better measuring for the presence of the tracking agent, layer, or insignia. By virtue of protocols having longitudinal character, at process 1720, the system cross-references data 1710 with previous measurements and desired outcomes, generating medication protocol adherence patterns (protocol) 1750 (e.g. for the particular patient, device, care facility, population group, etc.). The process 1720 detects missed intake or intake target for a particular medication and flags the detection. For example, such determination may be made by determining an amount of Al present or absent from an augmented utensil. The system also enables other types of protocol (pattern) deviation detection and analysis, so as to produce insights per patient, caretaking facilities, caretakers, type of medicine, type of disease being treated, co-morbidities, etc. In an example implementation, the system generates and may display an Adherence Dashboard 1760 that provides a projected patient regimen adherence score (as a percentage compliance in the illustrated example). In an example implementation, The system is configured to display information via the Adherence Dashboard 1760 at different levels, such as per facility, per patient within a given facility, per care-taker, etc. a facility level display can display percentage compliance for all augmented utensils fabricated and dispensed by a facility to different patients or to different caretakers. A patient level display can display adherence on a per patient basis. The caretaker level display can display adherence on a per caretaker basis.

In some examples, the process in FIG. 17 determines from the medication adherence protocol (pattern) whether an adjustment to the medication adherence protocol is to be made. For example, a process can access the amount of deviation from the medication adherence protocol or the frequency of deviation or the type of deviation and from that analysis determine if the medication adherence protocol is to be modified, in which case a redefinition protocol process 1740 is implemented and updated adherence protocol data is stored.

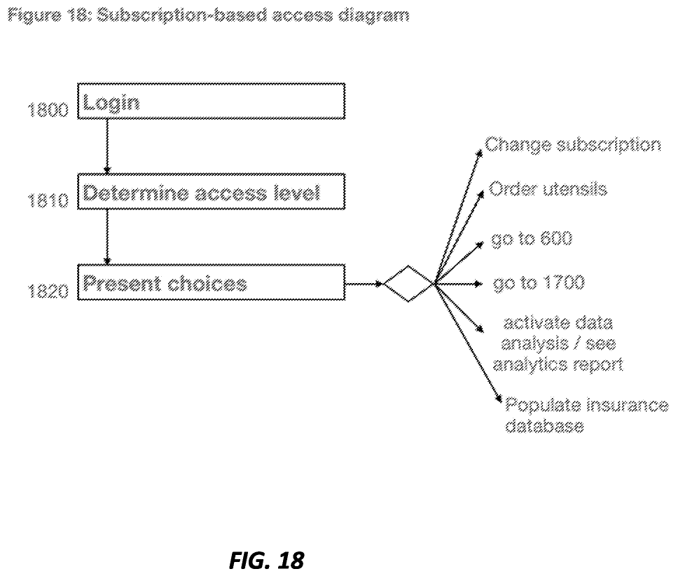

FIG. 18 illustrates an example subscription-based access method, in accordance with an example. In a process 1800, the user logs into the service, in some implementations, via web interface, via an app, etc. In process 1810, the system determines the level of access and functionality enabled by the system for the user logging in. The system then presents choice(s) to the user in process 1820 based on the determined level of access. Examples of choices include but are not limited to: changing the subscription, ordering additional utensils, activating the protocol modification process and system 600 (FIG. 6), displaying an adherence dashboard 1760 and/or raw or processed dataset 1750 (FIG. 17), generating a summary report, generating a report with optional recommendations following analysis by a physician and/or a deep learning algorithm, populating the insurance database with information on adherence, providing a marketplace for products and services associated with the data in the system.

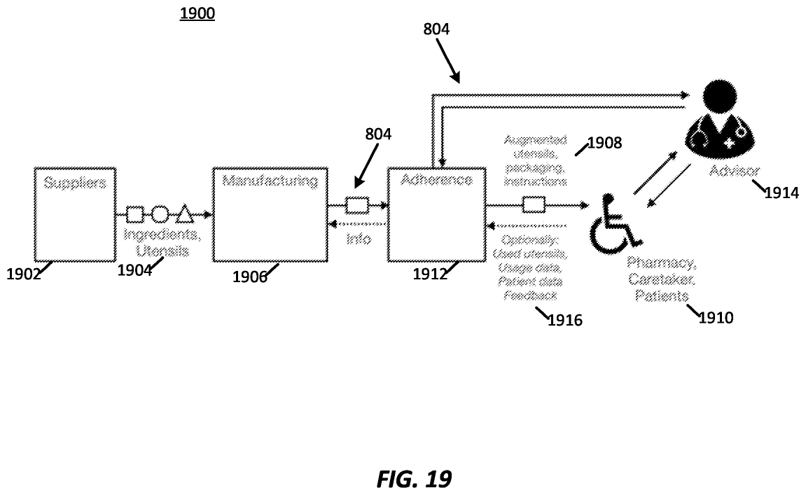

FIG. 19 illustrates an example implementation of a system 1900 implementing techniques herein, according to an example. Various suppliers 1902 of active ingredients and/or utensils provide their supplies 1904 to a manufacturing system 1906, where the augmented utensils are generated. In accordance with examples herein, these augmented utensils may incorporate among other things active pharmaceutical ingredients and/or supplements. In accordance with examples herein, manufacturing methods include but are not limited to, optionally, depending on cost and ingredient requirements: dip-coating, vacuum coating, spray-coating, ink-jet printing, organic vapor jet printing, chemical vapor deposition, sputtering, dip-pen coating, soaking, 3D printing (of inorganic, organic, and/or biological materials). These augmented utensils 1908 are distributed to users 1910, which include among others pharmacies, caretakers, and patients. An adherence system 1912 receives used utensils, usage data, patient data, feedback data, or other data (collectively 1914) from a patient, caretaker, advisor, pharmacy, etc. (collectively 1916) and determines medication protocol adherence in accordance with techniques herein. In some examples, the adherence system 1912 enables advisors, caretakers, and/or patients to order utensils, access associated services, in addition to collecting data for monitoring and improvement of adherence, in accordance with techniques herein.

### Example Methods of Augmented Utensil Fabrication

In various examples, the augmented utensils may be formed by various methods, including for example, methods of (a) forming a compound vapor; (b) passing the compound vapor through a nozzle oriented towards the substrate; (c) depositing the compound on the substrate as a solid; (d) displacing one of the nozzle or the substrate relative to the other; and (e) repeating steps (c) and (d) as required to achieve a desired deposition protocol.

In some examples, the methods provided herein may comprise forming an Al film or Al layered structure of an inorganic compound or an organic compound. In some cases, the methods provided herein comprise an organic compound. The methods provided herein can comprise an organic compound that is between 100 and 5000 g/mol. In embodiments, the organic compound can be between approximately 100 and 1000 g/mol, for example, about 100 g/mol, about 150 g/mol, about 200 g/mol, about 250 g/mol, about 300 g/mol, about 400 g/mol, about 500 g/mol, about 600 g/mol, about 700 g/mol, about 800 g/mol, or about 900 g/mol. In some cases, the organic compound of the disclosure herein can have a vapor pressure of 10⁻³ Pascals to 10⁶ Pascals, such as, 10⁻² Pascals to 105 Pascals, or 1 Pascals to 104 Pascals.

In some cases, the organic compound can comprise a drug, e.g., Al. As noted above, in embodiments, the Al can comprise a compound selected from the group consisting of caffeine, acetaminophen (paracetamol), carbamazepine, 5-methoxy sulfadiazine, ethenzamide, nalidixic acid, isoniazid, furosemide, sulfadimidine, celecoxib, temozolamide, piroxicam, tryptamine, chlorzoxazone, p-coumaric, itraconazole, fluoxetine, telaprevir, sildenafil, theophylline, aceclofenac, 5-nitrouracil, indomethacin, aripiprazole, and atorvastatin, or a mixture thereof.

In some cases, the organic compound is a supplement, excipient agent, or other active ingredient, but not an Al. In various embodiments, the organic compound can comprise an agricultural and/or food industry-relevant compound. In some cases, the compound can comprise a nutritional or food compound, a nutraceutical compound, a cosmetic or personal care compound, a fragrance compound, a colorant or dye, an ink, a paint, and the like, by way of non-limiting example. In some cases, the organic compound can comprise a pesticide, antibacterial, confectionary, seasoning, glaze or a mixture thereof. In some embodiments, the organic compound can comprise gibberellin, benzylideneacetone, or acesulfame.

The present disclosure thus provides for an augmented utensil having one or more active ingredient layers (also termed herein “solid films”). These active layers may be single compound structures or multiple-compound structures. These active layers may be formed of a single Al solid film layer, a single supplement solid film layer, a single excipient solid film layer, or some combination thereof. These active layers may be Al layered structures. In any event, these solid films herein may be formed, for example, of a deposited organic compound, such as a pharmaceutical active agent or a new chemical entity, patterned on a surface of a substrate of the utensil. In certain variations, the surface has a continuous surface coating or film of the organic compound, while in other variations, the organic compound may be applied to select discrete regions of the surface. High quality films or coatings of low molecular organic compounds are formed by the processes according to certain aspects of the present disclosure that have high purity levels. For example, in certain variations, a purity level in one or more regions where of the compound is deposited may be greater than or equal to about 90% by mass of the compound, optionally greater than or equal to about 95% by mass, optionally greater than or equal to about 97% by mass, optionally greater than or equal to about 98% by mass, and in preferred aspects, optionally greater than or equal to about 99% by mass, optionally greater than or equal to about 99.5% by mass, optionally greater than or equal to about 99.7% by mass, and in certain variations, greater than or equal to about 99.99% by mass purity concentration. In certain variations, multiple compounds are present that together or cumulatively have the same purity levels. These compounds may be any suitable active ingredient, including pharmaceutical ingredients or supplements. The deposited solid film may have a surface feature morphology ranging from molecularly flat to high surface area (e.g., a nanostructured surface) with feature sizes in the micrometer or nanometer regimes.

In certain aspects, methods of achieving solid films with high levels of purity and solubility are provided. For example, in certain variations, a solvent-free vapor deposition method is provided that includes depositing an organic compound on one or more discrete regions of a substrate in a process that is substantially free of solvents. By “substantially free” it is meant that solvent compounds or species are absent to the extent that undesirable and/or detrimental effects are negligible or nonexistent. In certain aspects, a vapor deposition process that is substantially free of solvents has less than or equal to about 0.5% by weight, optionally less than or equal to about 0.1% by weight, and in certain preferred aspects, 0% by weight of the undesired solvent species present during the deposition process.

A deposited organic compound may then be present at high purity levels, for example, at greater than or equal to about 99 mass % as described above, in the one or more discrete regions. The process for depositing the organic compound may be selected from the group consisting of, for example: dip-coating, spray-coating, ink-jet printing, organic vapor jet printing, chemical vapor deposition, sputtering, dip-pen coating, soaking, 3D printing (of inorganic, organic, and/or biological materials), vacuum thermal evaporation (VTE), organic vapor phase deposition (OVPD), organic molecular beam deposition (OMBD), molecular jet printing (MoJet), and organic vapor phase deposition (OVPD).

In certain aspects, such a method may include entraining the organic compound in an inert gas stream or vacuum that is substantially free of any solvents prior to the depositing. An inert gas stream can comprise one or more generally nonreactive compounds, such as nitrogen, argon, helium, and the like. In certain variations, the inert gas stream comprises nitrogen.

Because many organic compounds, such as small molecular medicines, have sufficiently high vapor pressures (e.g., from about 1 Pa to about 100 Pa) and relatively low evaporation enthalpies (e.g., 100-300 kJ/mole), high evaporation rates (on the order of grams/(sec*m²)) can be achieved at temperatures of 100°−500° C., without reaching the temperature range where degradation of the compound can occur, even when evaporating at atmospheric pressure. Any process/system that enables deposition of molecular material onto a substrate from a vapor phase, where a source of the molecular material is a solid that evaporates or sublimates, can be used for forming the deposited organic compound pharmaceutical substances. This includes, but is not limited to: vacuum thermal evaporation (VTE), organic vapor phase deposition (OVPD), organic molecular beam deposition (OMBD), and molecular jet printing (MoJet).

However, the processes are not limited to solid sources of the compound. In certain aspects, prior to the entraining, the organic compound is in a form selected from the group consisting of: a powder, a pressed pellet, a porous material, and a liquid, e.g., a solution of a compound. In certain aspects, prior to the entraining, the organic compound is dispersed in pores of a porous material. In other aspects, prior to the entraining, the organic compound is dispersed in a liquid bubbler through which the inert gas stream passes. In yet other aspects, the entraining of the organic compound in the inert gas stream or vacuum is conducted by heating a source of a solid organic compound to sublimate or evaporate the organic compound.

A parameter of the deposition process may be adjusted to control or affect a morphology, a degree of crystallinity, or both the morphology and the degree of crystallinity of the deposited solid organic compound. The parameter is selected from the group consisting of: system pressure, a flow rate of the inert gas stream, a composition of the inert gas, a temperature of a source of the organic compound, a composition of the substrate, a surface texture of the substrate, a temperature of the substrate, and combinations thereof.

In certain aspects, a specific surface area of the deposited organic compound is greater than or equal to about 0.001 m²/g to less than or equal to about 1,000 m²/g. The deposited organic compound may be amorphous. When the deposited organic compound is amorphous, it may further define interconnected particles having an average particle size (e.g., average particle diameter) of greater than or equal to about 2 nm to less than or equal to about 200 nm. In other aspects, the deposited organic compound is crystalline or polycrystalline. In such variations, an average crystal size or domain may be greater than or equal to about 2 nm to less than or equal to about 200 nm.

In certain aspects, the one or more discrete regions on which the organic compound is deposited are continuous so that a solid film is formed on the surface of the utensil or other substrate. In certain variations, the one or more discrete regions of the surface have a high surface area morphology, which may optionally define one or more nanostructures or microstructures. In certain variations, the films are flat (roughness <100 nm). In certain variations, the films comprise micro- and/or nano-structures.

In certain variations, a purity level of the deposited organic compound in the one or more discrete regions is any of those described previously, for example, greater than or equal to about 99.5 mass %. Suitable organic compounds, which may be pharmaceutical active ingredients or new chemical entities, may include by way of non-limiting example, various drugs or potential drugs (e.g., new chemical entities), including anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. The description of these suitable organic compounds/pharmaceutical active ingredients/new chemical entities is merely exemplary and should not be considered as limiting as to the scope of compounds or active ingredients which can be applied to a surface according to the present disclosure, as all suitable organic molecules and/or active ingredients known to those of skill in the art for these various types of compositions are contemplated. Furthermore, an organic compound may have various functionalities and thus, can be listed in an exemplary class above; however, may be categorized in several different classes of active ingredients.

Various suitable active ingredients are disclosed in Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and at http://www.drugbank.ca/, the relevant portions of each of which are incorporated herein by reference. Each additional reference cited or described herein is hereby expressly incorporated by reference in its respective entirety. In certain variations, the organic compound is an active ingredient compound selected from the group: caffeine, (E)-3-(4-Methylphenylsulfonyl)-2-propenenitrile, fluorescein, paracetamol, ibuprofen, tamoxifen, cannabidiol, and combinations thereof. BAY 11-7082 ((E)-3-(4-Methylphenylsulfonyl)-2-propenenitrile) selectively and irreversibly inhibits transcription factor NF-κB activation (which otherwise regulates expression of inflammatory cytokines, chemokines, immunoreceptors, and cell adhesion molecules) and can inhibit TNF-α-induced surface expression of adhesion molecules ICAM-1, VCAM-1, and E-selectin in human endothelial cells.

In certain variations, the deposited organic compound has an enhanced rate of dissolution in comparison to a comparative powder or pellet form of the same deposited organic compound. Thus, a dissolution rate of the deposited organic compound in an aqueous solution (e.g., approximating physiological conditions) is at least ten times greater than a comparative dissolution rate of the comparative powder or pellet form of the deposited organic compound. In certain variations, a dissolution rate of the deposited organic compound in an aqueous solution is at least fifteen times greater, optionally twenty times greater, and optionally thirty times greater than a comparative dissolution rate of the powder or pellet form of the deposited organic compound.

In certain aspects, a solid film having a high surface area morphology can be formed by a modified organic vapor jet printing (OVJP) process, which eliminates the need for organic solvents and improves dissolution rates for small molecular-based organic materials, like Als. The organic compound(s) that may be deposited by the OVJP process have relatively s and thus are considered to be organic compounds. OVJP processes utilize a carrier gas (e.g., nitrogen) to transport sublimated organic vapor towards a cooled substrate or other target in the form of a focused gas jet. The OVJP process enables scalable patterning of relatively small molecular materials.

In certain aspects, the present disclosure thus contemplates a solid film comprising greater than or equal to about 99 mass % of a deposited organic active ingredient compound having a molecular weight of less than or equal to about 1,000 g/mol. For example, the deposited organic compound may have a molecular weight of greater than or equal to about 100 g/mol to less than or equal to about 900 g/mol. The organic active ingredient compound is preferably a pharmaceutical active or a new chemical entity. The organic active ingredient is any of the compounds described above. By way of example, the deposited organic active ingredient compound may be selected from the group consisting of: anti-proliferative agents; anti-rejection drugs; anti-thrombotic agents; anti-coagulants; antioxidants; free radical scavengers; nucleic acids; saccharides; sugars; nutrients; hormones; cytotoxin; hormonal agonists; hormonal antagonists; inhibitors of hormone biosynthesis and processing; antigestagens; antiandrogens; anti-inflammatory agents; non-steroidal anti-inflammatory agents (NSAIDs); antimicrobial agents; antiviral agents; antifungal agents; antibiotics; chemotherapy agents; antineoplastic/anti-miotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; targeting toxin agents; and combinations thereof. In certain variations, the deposited organic active ingredient compound is selected from the group consisting of: caffeine, (E)-3-(4-Methylphenylsulfonyl)-2-propenenitrile, fluorescein, paracetamol, ibuprofen, tamoxifen, and combinations thereof.

In certain aspects, the solid film has a specific surface area of the solid film that is greater than or equal to about 0.001 m2/g to less than or equal to about 1,000 m2/g. In certain variations, the deposited organic active ingredient compound in the solid film is amorphous. The solid film may further define particles having an average particle size of greater than or equal to about 2 nm to less than or equal to about 200 nm. Where the solid film is amorphous, the deposited organic active ingredient compound in the solid film is stable for greater than or equal to about 1 month, optionally greater than or equal to about 2 months, optionally greater than or equal to about 3 months, optionally greater than or equal to about 6 months, optionally greater than or equal to about 9 months, and in certain variations, optionally greater than or equal to about 1 year.

In other variations, the deposited organic active ingredient compound in the solid film is crystalline or polycrystalline. An average crystal size may be greater than or equal to about 2 nm to less than or equal to about 200 nm. An average thickness of the solid film may be less than or equal to about 300 nm and an average surface roughness (Ra) of the solid film is less than or equal to about 100 nm.

In other variations, an average thickness of the solid film is greater than or equal to about 300 nm. An average surface roughness (Ra) is greater than or equal to about 100 nm. The film having such a thickness defines a nanostructured surface comprising a plurality of nanostructures having a major dimension of greater than or equal to about 5 nm to less than or equal to about 10 μm. In such an embodiment, the plurality of nanostructures may have a shape selected from the group consisting of: needles, tubes, rods, platelets, round particles, droplets, fronds, tree-like structures, fractals, hemispheres, puddles, interconnected puddles, islands, interconnected islands, and combinations thereof.

Source temperature can be determined via thermogravimetry and tuned to obtain local deposition rate of approximately 0.5 μg/min. The temperature range and carrier gas rate can change depending on system size and configuration.

In certain embodiments, the solid film may comprise a deposited organic compound comprising caffeine. The plurality of nanostructures can have a particular structure, e.g., a needle shape or a tube shape. An average diameter of the plurality of nanostructures can be greater than or equal to about 5 nm to less than or equal to about 10 μm and an average length of greater than or equal to about 5 nm to less than or equal to about 100 μm.

In certain other embodiments, the solid film may comprise a deposited organic compound comprising (E)-3-(4-Methylphenylsulfonyl)-2-propenenitrile (BAY 11-7082). The plurality of nanostructures has a platelet shape, where an average height of the plurality of nanostructures is greater than or equal to about 10 nm to less than or equal to about 10 μm. An average width of the plurality of nanostructures is greater than or equal to about 5 nm to less than or equal to about 10 μm. An average length of greater than or equal to about 5 nm to less than or equal to about 100 μm.

In yet other embodiments, the solid film may comprise a deposited organic compound comprising fluorescein.

In yet further embodiments, the solid film may comprise a deposited organic compound comprising paracetamol. The plurality of nanostructures has a shape selected from the group consisting of: droplet, hemisphere, puddle, interconnected puddle, island, interconnected island, and combinations thereof, wherein an average major dimension of the plurality of nanostructures is greater than or equal to about 5 nm to less than or equal to about 20 μm.

In other aspects, the deposited organic compound according to the present teachings has an enhanced rate of dissolution as compared to a comparative powder or pellet form of the organic active ingredient. A dissolution rate of the deposited organic active ingredient compound in the solid film in an aqueous solution is at least ten times greater than a comparative dissolution rate of the comparative powder or pellet form of the organic active ingredient. The dissolution rate improvement may be any of those previously discussed above.

The films disclosed herein also have improved stability as compared to a comparative film of the active ingredient. In some cases, the films are substantially free of water or other solvents, reducing the rate and extent of degradation of the film.

In certain aspects, the solid film is substantially free of any binders or impurities. A solid film that is substantially free of binders or impurities has less than or equal to about 0.5% by weight, optionally less than or equal to about 0.1% by weight, and in certain preferred aspects, 0% by weight of the undesired binders or impurities present in the solid film composition. In certain variations, the solid film comprises greater than or equal to about 99.5 mass % of the deposited organic active ingredient compound; however, any of the purity levels discussed above may likewise be achieved in the solid film.

In certain aspects, the deposited organic compound on the surface is crystalline or polycrystalline. In other aspects, the deposited organic compound is amorphous. In this manner, substantially pure molecular medicinal films are fabricated that may have high surface area morphologies. The deposited organic compound exhibits enhanced solubility and stability.

In other variations, the present disclosure contemplates a solid film comprising multiple deposited organic active ingredient compounds each having a molecular weight of less than or equal to about 1,000 g/mol. The organic active ingredient compounds are preferably a pharmaceutical active or a new chemical entity. The organic active ingredient compounds are any of the compounds described above. A collective amount of the multiple organic active ingredient compounds may be greater than or equal to about 99 mass % in the solid film. The solid films may have any of the compositions or features described just above, which will not be repeated herein for brevity.

The methods provided herein can comprise subliming the compound to form a compound vapor. In some cases, the organic compound is sublimed at a temperature that is 1° C. to 300° C. above its onset of sublimation, as determined by thermogravimetric analysis (“TGA”), for example, 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 25° C., 30° C., 35° C., 40° C., 45° C., 50° C., 60° C., 70° C., 80° C., 90° C., 100° C., 150° C., 200° C., 250° C., or 300° C. above its onset of sublimation. In some cases, the organic compound is sublimed at a temperature that is 5° C. to 150° C., 10° C. to 100° C., or 10° C. to 50° C. above its onset of sublimation, as determined by TGA. In some embodiments, the compound vapor is supersaturated. In various cases, the compound vapor is in the presence of a carrier gas.

A carrier gas can comprise an inert gas (e.g., nitrogen, argon, CO₂, etc.) or a reactive gas (e.g. HCl, H₂O, O₃, CH₄, O₂ etc.). In some cases, the carrier gas can comprise nitrogen, carbon dioxide, krypton, argon, hydrogen, helium, oxygen, water, methane, nitrous oxide or a mixture thereof. In some cases, the carrier gas has a flow rate of about 1 to 500 standard cubic centimeters per minute (“sccm”). In embodiments, the carrier gas can have a flow rate of about 1 to 200 sccm, such as, about 1 to 150 sccm, about 1 to 100 sccm, about 1 to 50 sccm, or about 25 to 50 sccm, for example, 1 sccm, 5 sccm, 10 sccm, 15 sccm, 20 sccm, 25 sccm, 30 sccm, 35 sccm, 40 sccm, 45 sccm, 50 sccm, 60 sccm, 70 sccm, 80 sccm, or 90 sccm. In various embodiments, the carrier gas can carry the organic compound to a mixing chamber.

In some cases, the method provided herein can further comprise exposing the carrier gas to a guard force gas, such that the guard force gas surrounds the carrier gas, and the carrier gas comprises the vapor mixture. In some embodiments, the guard force gas comprises nitrogen, carbon dioxide, kypton, argon, hydrogen, helium, methane, nitrous oxide, or a mixture thereof.

The methods provided herein comprise condensing the vapor mixture onto a substrate to form the film. In embodiments, the method herein can further comprise moving the vapor mixture over the substrate. In some embodiments, the nozzle can be rastered over the substrate such that the vapor mixture moving over the substrate. In some embodiments, the nozzle can be rastered keeping the center to center line spacing from 0.01 mm to 100 mm, such as, 0.01 mm, 0.05 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, or 10 mm. In various embodiments, the nozzle can be rastered such that the raster velocity is from 0.01 mm/s to 100 mm/s. In some embodiments, the raster velocity is from 0.01 mm/s to 10 mm/s, for example, 0.01 mm/s, 0.05 mm/s, 0.1 mm/s, 0.2 mm/s, 0.3 mm/s, 0.4 mm/s, 0.5 mm/s, 0.6 mm/s, 0.7 mm/s, 0.8 mm/s, 0.9 mm/s, 1 mm/s, 2 mm/s, 3 mm/s, 4 mm/s, 5 mm/s, 6 mm/s, 7 mm/s, 8 mm/s, 9 mm/s, or 10 mm/s. In some cases, the method can further comprise moving the substrate under the vapor mixture.

In some embodiments, the substrate can be at a temperature of −100° C. to 100° C., for example, −90° C., −80° C., −70° C., −60° C., −50° C., −40° C., −30° C., −20° C., −10° C., −5° C., 0° C., 5° C., 10° C., 15° C., 20° C., 25° C., 30° C., 35° C., 40° C., 45° C., 50° C., 55° C., 60° C., 65° C., 75° C., 80° C., 85° C., 90° C., or 95° C. In some cases, the substrate can be at a temperature of −50° C. to 50° C., such as, 5° C. to 25° C. The substrate can be made of any material that one of skill in the art would find suitable to coat with the film.

As used herein, the term “substrate” refers to substances that can be used to form a utensil, in particular a working end of the utensil, including but not limited to metal, plastic, wood, ceramic, quartz, glass, paper, composites, or the like, or a mixture thereof, or an aqueous or non-aqueous solvent, e.g., an organic solvent. In embodiments, the substrate can comprise quartz, glass, metal, plastic, ceramic or a combination thereof.

### Printed Augmented Utensils

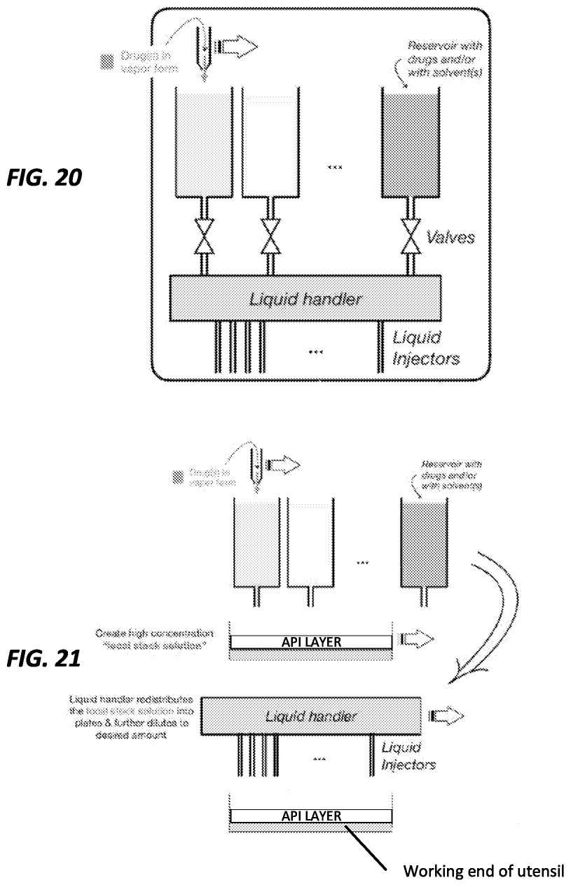

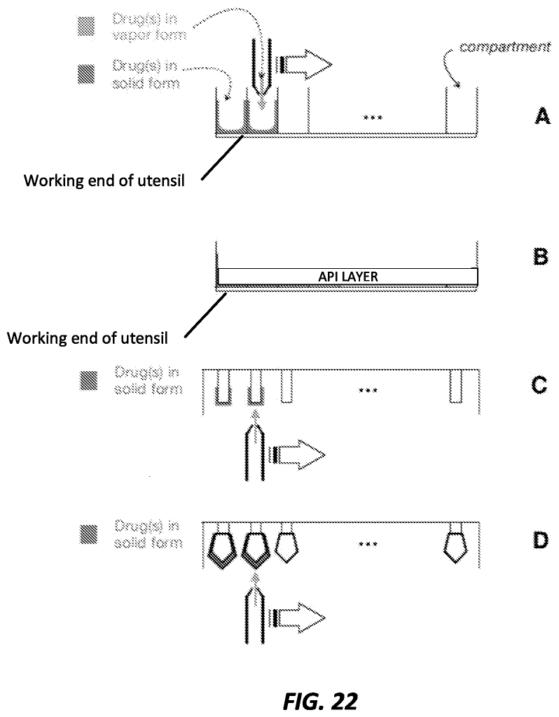

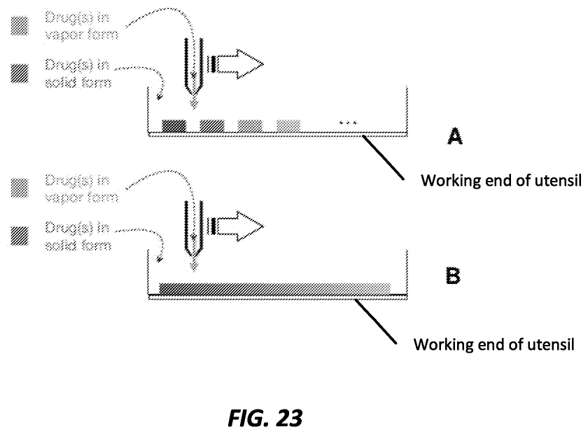

Also described herein are fabricated augmented utensils comprising a solid deposited film formed on a utensil to create an augmented utensil, where that solid film may comprise an Al such, as, an organic compound or a drug, or a pharmaceutical composition comprising at least one organic compound. The solid deposited films may have any of the composition or features described above. The augmented utensils can be prepared as in FIGS. 20-23.

In some cases, an augmented utensil is provided that includes a surface of a solid substrate having one or more discrete regions patterned with a deposited organic compound having a molecular weight of less than or equal to about 1,000 g/mol. The organic compound may be any suitable active ingredient including any of the compounds described above. In some examples, the deposited organic compound that forms the active ingredient layer is present at greater than or equal to about 99 mass % in the one or more discrete regions. In some examples, an Al layered structure is used with an Al mixed in a ratio with another compound. In some examples, the purity of the deposited compound will be limited by the starting material, which in some augmented utensil applications can be lower than 99%. In certain aspects, the one or more discrete regions of the surface are continuous and the deposited solid organic compound forms a solid film on the surface of the pharmaceutically acceptable substrate.

In other cases, an augmented utensil is provided that includes a surface of a solid substrate having one or more discrete regions patterned with multiple deposited organic compounds each having a molecular weight of less than or equal to about 1,000 g/mol. The organic compounds are any of the compounds described above. The multiple deposited organic compounds are cumulatively present at greater than or equal to about 99 mass % in the one or more discrete regions. Thus, any of the solid films described above may be disposed on a surface of a solid substrate. Further, the solid substrate may be as described just above.

In some examples, the augmented utensil is formed of a pharmaceutically acceptable substrate defining a surface. The materials selected for the substrate are preferably pharmaceutically acceptable or biocompatible, in other words, substantially non-toxic to cells and tissue of living organisms. Pharmaceutically acceptable materials may be those which are suitable for use in contact with the tissues of humans and other animals without resulting in excessive toxicity, irritation, allergic response, or other problems or complications, commensurate with a reasonable benefit/risk ratio.

In certain aspects, the pharmaceutically acceptable substrate is biodegradable. By biodegradable, it is meant that the materials forming the substrate dissolve or erode upon exposure to a solvent comprising a high concentration of water, such as serum, growth or culture media, blood, bodily fluids, or saliva.

In some examples (such as an example OVJP), the present techniques include an application stage, e.g., jet printing or deposition, optionally followed by reorientation of a substrate relative to a printing means such as a nozzle, and repetition until a desired pattern is obtained to form the augmented utensil having an Al layered structure. The application stage may be part of the augmented utensil provider 112, for example. An application stage may operate using a customized cartridge, such as a dynamic cartridge that provides a local solution intended for use in the near-term. The distribution and application stages can be selected based on the form and nature of the distribution of application. For example, the distribution and application stages can be appropriately modified if a compound is vapor jetted into a liquid or directly onto a surface to provide a film. An application stage may include a module for vapor jetting drug or imaging molecules. Such a module can comprise a source of a compound, heaters to vaporize the compound, a carrier gas inlet and flow channel, a temperature probe, and a mounting plate and tip holder to maintain the module in proper position for control of the vapor jetting application.

The foregoing description is given for clarity of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of several of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

We claim:

1. A system for tracking patient medication protocol adherence, the system comprising:
a computing device having one or more processors and one or more memories, the computing device communicatively coupled to a communication network, the computing device being configured to allow one or more remote user devices to access the computing device via the communication network;
the computing device being further configured to,
provide an interface of an application to the one or more remote user devices,
receive, via the interface, identification data on a subject, the identification data including a pathology data for the subject,
determine from the identification data a desired medication protocol for the subject, and
convert the desired medication protocol into augmented utensil configuration data for the subject, the augmented utensil configuration data comprising (i) a desired amount of an active pharmaceutical ingredient (Al) to be administered to the subject using an augmented utensil comprising a working end having layered Al delivery structure formed thereon and (ii) augmented utensil data and/or usage data and/or patient data containing at least one parameter of the augmented utensil;
the computing device being communicatively coupled, through the communication network, to a detection stage configured to analyze the augmented utensil and detect an amount of used Al,
the computing device being further configured to,
receive from the detection stage the amount of used Al and compare the amount of used Al to the desired amount of the Al stored in the augmented utensil configuration data, and
from the comparison of the amount of used Al to the desired amount of the Al, determine an adherence score indicating a predicted adherence of the subject to the medication protocol.

2. The system of claim 1, wherein the identification data comprises usage pattern, time of day, location, one or more Als to be taken, a medication delivery time frame for each of the one or more Als, preferred taste profile(s), counterindications, co-prescriptions, dosage, expiration dating information, storage instructions, safety instructions, prescribed regimen, regimen starting time and date, regimen ending time and date, patient gender, body weight, height, body mass index, place of administration, manner of administration, administration restrictions, temperature sensitive APIs restricted from certain utensils, preferred foods, counter-indicated foods, person responsible for administration, patient address, service subscription information, and/or service renewal information.

3. The system of claim 1, wherein the augmented utensil data identifies a number of layers of an Al layered structure data of the utensil, identifies one or more excipient layers of the utensil, identifies a flavor masking layer of the utensil, identifies a delivery delay layer of the utensil, identifies a tracking ingredient of the utensil, identifies a non-API material of utensil, and identifies a pattern of locations of the Al on the utensil.

4. The system of claim 1, wherein the augmented utensil data identifies a shape of a working end of the utensil.

5. The system of claim 1, wherein the augmented utensil data identifies a location of the API on a working end of the utensil.

6. The system of claim 1, wherein the computing device is further configured to compare the amount of used Al to the desired amount of the Al stored using a medication protocol model including one or more data elements of the augmented utensil configuration data.

7. The system of claim 1, wherein the one or more remote user devices comprises a patient user device, and the computing device is configured to authenticate the patient user device.

8. The system of claim 1, wherein the one or more remote user devices comprises a prescriber user device, and the computing device is configured to authenticate the prescriber user device.

9. The system of claim 1, wherein the computing device is configured to convert the desired medication protocol into augmented utensil configuration data for the subject:
by accessing a listing of available augmented utensil models each having a different augmented utensil configuration data;
ranking the available augmented utensil models according to a list of patient preferences, caretaker preferences, patient history, popularity across relevant demographic groups, preferred foods, preferred drinks, preferred administration methods, and dosage; and
identifying a highest ranked available augmented utensil model.

10. The system of claim 1, wherein the computing device is configured to perform a validation on the detection stage to confirm the augmented utensil analyzed by the detection stage corresponds to the augmented utensil data.

11. The system of claim 1, wherein the computing device is configured to send a report of the adherence score to an external computing device and/or store the adherence score as patient data, facility data, and/or caretaker data.

12. A method of customizing drug delivery, the method comprising:
receiving, at one or more processors, a drug delivery regimen profile for a subject, wherein the drug delivery regimen profile comprises at least one of one or more instances of usage pattern, time of day, location, one or more Is to be taken, a medication delivery time frame for each of the one or more Als, preferred taste profile(s), counterindications, co-prescriptions, dosage, expiration dating information, storage instructions, safety instructions, prescribed regimen, regimen starting time and date, regimen ending time and date, patient gender, body weight, height, body mass index, place of administration, manner of administration, administration restrictions, temperature sensitive Als restricted from certain utensils, preferred foods, counter-indicated foods, person responsible for administration, patient address, service subscription information, and/or service renewal information;
accessing a database identifying a plurality of available active pharmaceutical ingredients (Als) and excipients, with corresponding information regarding the molecular weight of the Al, chemical structure, chemical database reference number(s), solubility, enthalpy of vaporization, vapor pressure, melting point, thermal decomposition point, viscosity, hardness, taste profile, counterindications, co-prescriptions, dosage, expiration dating information, storage instructions, safety instructions, associated billing codes, prescribed regimen for a given patient, regimen starting time and date, regimen ending time and date, patient gender, body weight, height, body mass index, genetic information, place of administration, manner of administration, administration restrictions, temperature sensitive APIs restricted from certain utensils, preferred foods, counter-indicated foods, person responsible for administration, patient address, service subscription information, and/or service renewal information;
determining from the drug delivery regimen profile and the database an Al delivery structure comprising one or more of the available active Als and/or excipients;
determining a configuration and/or Al pattern based on the drug delivery regimen; and
depositing the Al delivery structure to a working end of a utensil having an elongated handle, the Al delivery structure being deposited according to the configuration and/or Al pattern.

13. A system for tracking patient medication protocol adherence, the system comprising:
a utensil to which the active ingredient (Al), optionally combined with excipient(s), is directly or indirectly affixed or coated or layered onto, and subsequently removable from, enabling direct oral delivery of Al to the subject, as the subject may eat or drink using said utensil;
a detection stage configured to receive the utensil and detect a usage measurement of and/or from the utensil;
one or more processors; and
one or more memories storing instructions that, when executed, cause the one or processors to:
determine a desired amount of the Al to be provided to the subject orally based on subject profile data;
determine an initial amount of the Al on the utensil;
determine, from the usage measurement, an actual amount of the Al remaining on the utensil or an actual amount of the Al removed from the utensil; and
determine a projected patient regimen adherence from a comparison of the actual amount of the Al remaining on the utensil or an actual amount of the Al removed from the utensil and the desired amount of the Al to be provided to the subject.

14. The system of claim 13, wherein the utensil comprises: an elongated handle end to be held in place during provision of the API to the subject; and a working end coupled to and extending from the handle end, the working end shaped to deliver the Al to the subject orally, the working end having surface onto which, directly or indirectly, the Al, and optionally the excipient(s), is or coated or layered,

15. The system of claim 13, wherein the utensil is a container.

16. The system of claim 13, wherein the utensil is a fork, spoon, blade chopstick, cup, sippy cup, lid of cup, lid of sippy cup, or lid of a reusable water bottle.

17. A utensil comprising:
an active ingredient (Al), optionally combined with excipient(s), directly or indirectly affixed or coated or layered onto, and subsequently removable from, a body of the utensil enabling direct oral delivery of Al to subject, as the subject may eat or drink using said utensil.

18. The utensil of claim 17, wherein the utensil comprises: an elongated handle end to be held in place during provision of the API to the subject; and a working end coupled to and extending from the handle end, the working end shaped to deliver the Al to the subject orally, the working end having surface onto which, directly or indirectly, the Al, and optionally the excipient(s), is or coated or layered,

19. The utensil of claim 17, wherein the utensil is a container.

20. The utensil of claim 17, wherein the utensil is a fork, spoon, blade, chopstick, cup, sippy cup, lid of cup, lid of sippy cup, or lid of a reusable water bottle.

21. The utensil of claim 17, further comprising a layered Al delivery structure comprising the Al and at least one excipient layer for affecting kinetics of provision, and/or pharmacokinetics, and/or bioavailability, of the Al to the subject orally.

22. The utensil of claim 17, further comprising a layered Al delivery structure comprising the Al and at least one excipient layer comprising a flavor masking layer film comprising a flavor masking agent for altering a perceived taste of the Al during provision of the Al to the subject orally.

23. The utensil of claim 17, further comprising a layered Al delivery structure further comprising the Al and at least one excipient layer comprising a delivery delay layer film comprising a delaying agent for delaying absorption of the Al to the subject after provision of the Al to the subject orally.

24. The utensil of claim 17, further comprising a layered Al delivery structure further comprising that Al and at least one excipient layer comprising a tracking ingredient separate from the Al, an amount of the tracking ingredient associated with the utensil in the second state, and thus after use by the subject, being indicative of an amount of the Al used by the subject.

25. The utensil of any of claims 21-24, wherein the at least one excipient layer is adjacent to the Al film layer.

26. The utensil of any of claims 21-24, wherein the Al film layer is deposited on one of a first surface and a second, opposing surface of the utensil and the at least one excipient layer is deposited on the other of the first surface and the second surface of the utensil.

27. The utensil of any of claims 21-24, wherein the at least one excipient layer comprises at least one of a flavor masking agent, a delaying agent, a texturing agent, a moisture responsive agent, a coloring agent, a temperature responsive agent, a delivery delay layer film, adhesion control layer, and a protective layer.

28. The utensil of any of claims 21-24, wherein the layered Al delivery structure further comprises an indicator layer comprising a color indicator or symbology indicator, the indicator layer being at least partially covered by the Al film layer such that upon provision of the Al to the subject orally, the indicator layer becomes exposed or further exposed.

29. The utensil of any of claims 21-24, wherein the layered Al delivery structure further comprises a tracking layer that produces a signal when removed from the utensil or ingested by the subject.

30. The utensil of any of claims 21-24, wherein the layered Al delivery structure further comprises a tracking layer that in a first state of the utensil is prevented from detection by the Al delivery structure and that in a second state of the utensil corresponding to removal of at least a portion of the Al is detectable.

31. The utensil of claim 17, further comprising a layered Al delivery structure comprising a plurality of solid Al film layers, each solid Al film layer having a different Al.

32. The utensil of claim 17, further comprising a layered Al delivery structure comprising a plurality of solid Al film layers, each solid Al film layer having a different cross-sectional thickness than at least one other solid Al film layers.

33. The utensil of claim 17, further comprising a layered Al delivery structure comprising the Al and a non-Al material.

34. The utensil of claim 33, wherein the layered Al delivery structure comprises an Al film layer having the Al at patterned locations of the Al film layer and a non-Al material between the patterned locations.

35. The utensil of claim 34, wherein the Al is encapsulated in the Al film layer by a nano-capsule or micro-capsule at the patterned locations.

36. The utensil of claim 34, wherein the Al film layer comprises the Al encapsulated by one or more nano-capsules or micro-capsules.

37. The utensil of claim 17, wherein the utensil is a spoon, a fork, a blade, or a combination thereof.

38. The utensil of claim 17, wherein the utensil is a container having a lid, the utensil further comprising a layered Al delivery structure layered or deposited on the lid of the container, such as a cup or sippy cup lid, or the cup itself, or the leading end of a container or utensil.

39. The utensil of claim 17, further comprising a layered Al delivery structure layered or deposited on a leading edge of the utensil.

40. The utensil of claim 17, further comprising a layered Al delivery structure comprising small-molecule pharmaceutical ingredients, biologics, proteins, antibodies, excipients, gels, polymers, and/or inactive ingredients.

41. The utensil of claim 40, wherein the layered Al delivery structure comprises a three-dimensional (3D) printed biologic layered or deposited on the utensil.

42. The utensil of claim 17, further comprising imprinted or labeled on the utensil Al identification data, subject identification data, drug regimen dosage identification data, and/or subject specific instruction data.

43. The utensil of claim 17, wherein the Al is selected from the group consisting of an active pharmaceutical agent, a supplement, and a placebo.

44. A utensil usage stage comprising:
a housing;
a sample area for positioning at least the working end of the utensil of claim 1 for inspection;
a detection stage positioned to detect a usage indication signal from the utensil; and
a processing stage comprising at least one processor and at least one memory, the processing stage being configured to determine from the usage indication signal detected by the detection stage at least one of absence of the Al on the utensil, presence of the Al on the utensil, an amount of Al absent from the utensil, an amount of Al present on the utensil.

45. The utensil usage stage of claim 44, further comprising:
an illumination activation stage for illuminating the utensil in the sample area with a detection illumination,
wherein the detection stage comprises a photodetector for detecting the usage indication signal.

46. The utensil usage stage of claim 44, further comprising:
a thermal activation stage for applying a temperature change to the utensil in the sample area,
wherein the detection stage comprises a thermal detector for detecting the usage indication signal.

47. The utensil usage stage of claim 44, further comprising:
a mount in the sample area configured to hold the elongated handled end and/or the working end.
